(19) United States
(12) Patent Application Publication
(10) Pub. No.: US 2008/0050734 A1
(43) Pub. Date: Feb. 28, 2008

(54) Novel Cdk11 molecules and uses thereof

(75) Inventors: Steven R. Coats, Camarillo, CA (US); Peter S. Yakowec, Thousand Oaks, CA (US); Marc Payton, Newbury Park, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMGEN INC., Thousand Oaks, CA (US)

(21) Appl. No.: 11/698,694

(22) Filed: Jan. 26, 2007

Related U.S. Application Data
(63) Continuation of application No. 09/953,102, filed on Sep. 12, 2001, now abandoned.
(60) Provisional application No. 60/232,133, filed on Sep. 13, 2000.

(51) Int. Cl. C12Q 1/68; G01N 33/00

(52) U.S. Cl. 435/6; 436/86

(57) ABSTRACT

The present invention relates to a novel serine threonine kinase. The invention also relates to vector, host cells, antibodies and recombinant methods for producing the Cdk11 polypeptide. In addition, the invention discloses therapeutic, diagnostic and research utilities for Cdk11 and related products.

### RELATED APPLICATION

The present application claims priority benefit of U.S. Provisional Application No. 60/232,133 filed Sep. 13, 2000, which is incorporated herein by reference in its entirety.

### FIELD OF THE INVENTION

The present invention relates to a novel human cyclin dependent kinase 11 (Cdk11), and uses thereof. The invention also relates to vectors, host cells, selective binding agents, such as antibodies and/or cofactors, modulators of Cdk11 activity/function, and methods for producing Cdk11 polypeptides. Also provided for are methods for the diagnosis, treatment, amelioration and/or prevention of diseases associated with Cdk11 polypeptides.

### BACKGROUND OF THE INVENTION

Technical advances in identification, cloning, expression and manipulation of nucleic acid molecules and deciphering of the human genome have greatly accelerated discovery of novel therapeutics based upon deciphering of the human genome. Rapid nucleic acid sequencing techniques can now generate sequence information at unprecedented rates and, coupled with computational analyses, allow the assembly of overlapping sequences into the partial and entire genomes as well as identification of polypeptide-encoding regions. A comparison of a predicted amino acid sequence against a database compilation of known amino acid sequences allows one to determine the extent of homology to previously identified sequences and/or structural landmarks. The cloning and expression of a polypeptide-encoding region of a nucleic acid molecule provides a polypeptide product for structural and functional analyses, and provides a basis of identifying molecules that manipulate the polypeptide biological activity. The manipulation of nucleic acid molecules and encoded polypeptides to create variants and derivatives thereof may confer advantageous properties on a product for use as a therapeutic.

In spite of significant technical advances in genome research over the past decade, the potential for development of novel therapeutics based on the human genome is still largely unrealized. Many genes encoding potentially beneficial polypeptide therapeutics, or those encoding polypeptides which may act as “targets” for therapeutic molecules, have still not been identified.

Accordingly, it is an object of the invention to identify novel polypeptides and nucleic acid molecules encoding the same which have diagnostic or therapeutic benefit.

Cyclin-dependent kinases (cdks) are serine-threonine kinases primarily involved in cell cycle progression, maintenance of normal cell growth and gene transcription. Cdks are typically 300 amino acids in length and are characterized by their ability to bind members of the cyclin family. In addition, all cdks are activated by phosphorylation and have conserved protein motifs such as the T loop and the PSTAIRE motif.

Cyclins are cdk binding partners which contain a cyclin box domain. This 100 amino acid domain mediates cyclin interaction with a cdk (See McDonald and El-Deiry, Int. J. Oncolog., 16: 871-886, 2000). The interaction between the cdk and its cyclin binding partner forms a cdk holoenzyme wherein the cdk is the catalytic subunit. This holoenzyme is activated by phosphorylation of a conserved Thr within the catalytic domain. The kinase activity of the cdk-cyclin complex is stoichiometricly regulated by small inhibitory proteins known as cdk inhibitors, such as p21, p17, p27, p57, p16, p19 and p15. (See Senderowicz and Sausville, J.N.C.I., 92: 376-387, 2000). Phosphorylation at specific cdk amino acid residues will also inhibit cdk function such as the inhibitory phosphorylation of Thr14 and Tyr15 within cdk1. Specificity of cyclin binding is dictated by the 16 amino acid PSTAIRE domain of the cdk. The cyclin binding site is within the mid portion of the cdk PSTAIRE domain. (See McDonald and El-Deiry, Int. J. Oncolog., 16: 871-886, 2000).

Cdk activation is limited to the cdk-cyclin complex. The characteristic T loop domain is an 18 amino acid motif which contains the Thr phosphorylation site which activates the complex. In the unbound cdk, the T loop blocks the catalytic cleft and makes it inaccessible to potential substrates. Upon cyclin binding, the T loop shifts away from the catalytic subunit which allows the cleft to open and be available for binding substrates. (See McDonald and El-Deiry, Int. J. Oncolog., 16: 871-886, 2000).

Cdks play a role in regulating cellular progression through S phase and mitosis, and therefore, hyperactivation of cdks will result in uncontrolled cell growth. Loss of cell cycle regulation has been implicated in cancer progression and may play a role in other hyperproliferative pathological conditions. Cdk hyperactivation may be caused by an increase in transcription and/or translation of cdk molecules or mutations in cdk genes that cause constitutive phosphorylation of the activating Thr. Further, increased expression of other positive regulators (such as cyclins) and decreased expression of cdk inhibitors may cause hyperactivation of cdks. (See Senderowicz and Sausville, J.N.C.I., 92: 376-387, 2000).

Positive cell cycle regulators, such as cdks and cyclins, are known to be overexpressed in breast cancer, colorectal cancers, prostate cancer, brain tumors, melanoma, lymphomas, parathyroid adenoma, and leukemia. In addition, decreased levels of the cdk inhibitor protein, p27, correlate with poor prognosis in patients suffering from colon, gastric, breast and prostate tumors. Mice which have the p27 gene knocked out develop mulitorgan hyperplasia. Further, the gene which encodes the cdk inhibitor p16 is inactivated in familial and sporadic melanomas, pancreatic adenocarcinomas, and lung and bladder carcinomas. Deletions of the p19 cdk inhibitor gene have been identified in melanoma cell lines, gliomas and in acute T cell lymphoma. Accordingly mice which have the p19 gene knocked out exhibit a high incidence of spontaneous and induced tumors. (See Funk, Anticancer Research, 19: 4772-4780, 1999). A Cdk10 molecule has been disclosed in U.S. Pat. No. 5,986,800 (assigned to Merck & Co.) which is purported to be involved in cell cycle control.

Thus, identification of novel cyclin dependent kinases has led to a better understanding of cell cycle progression including cell proliferation and differentiation. Identification of the novel Cdk11 gene and polypeptide, as described herein, will further clarify the understanding of these processes and facilitate the development of therapies for pathological conditions which involve cellular hyperproliferation and other biological processes.

### SUMMARY OF THE INVENTION

The present invention relates to a novel serine/threonine kinase family and uses thereof. More specifically, the present invention relates to novel Cdk11 nucleic acid molecules and encoded polypeptides, and uses thereof.

The invention provides for an isolated nucleic acid molecule comprising a nucleotide sequence selected from the group consisting of:

(a) the nucleotide sequence set forth in SEQ ID NO: 1;

(b) a nucleotide sequence encoding the polypeptide set forth in SEQ ID NO: 2;

(c) a nucleotide sequence which hybridizes under moderately or highly stringent conditions to the complement of (a) or (b), wherein the encoded polypeptide has an activity of the polypeptide set forth in SEQ ID NO: 2; and

(d) a nucleotide sequence complementary to any of (a) through (c).

The invention also provides for an isolated nucleic acid molecule comprising a nucleotide sequence selected from the group consisting of:

(a) a nucleotide sequence encoding a polypeptide that is at least about 70, 75, 80, 85, 90, 95, 96, 97, 98, or 99 percent identical to the polypeptide set forth in SEQ ID NO: 2, wherein the polypeptide has an activity of the encoded polypeptide set forth in SEQ ID NO: 2 as determined using a computer program selected from the group consisting of GAP, BLASTP, BLASTN, FASTA, BLASTA, BLASTX, BestFit, and the Smith-Waterman algorithm;

(b) a nucleotide sequence encoding an allelic variant or splice variant of the nucleotide sequence set forth in SEQ ID NO: 1, wherein the encoded polypeptide has an activity of the polypeptide set forth in SEQ ID NO: 2;

(c) a nucleotide sequence of SEQ ID NO: 1, (a), or (b) encoding a polypeptide fragment of at least about 25 amino acid residues, wherein the polypeptide has an activity of the polypeptide set forth in SEQ ID NO: 2;

(d) a nucleotide sequence encoding a polypeptide that has a substitution and/or deletion of 1 to 346 amino acid residues set forth in any of SEQ ID NOS: 1-2 wherein the encoded polypeptide has an activity of the polypeptide set forth in SEQ ID NO: 2;

(e) a nucleotide sequence of SEQ ID NO: 1, or (a)-(d) comprising a fragment of at least about 16 nucleotides;

(f) a nucleotide sequence which hybridizes under moderately or highly stringent conditions to the complement of any of (a)-(e), wherein the encoded polypeptide has an activity of the polypeptide set forth in SEQ ID NO: 2; and

(g) a nucleotide sequence complementary to any of (a)-(e).

The invention further provides for an isolated nucleic acid molecule comprising a nucleotide sequence selected from the group consisting of:

(a) a nucleotide sequence encoding a polypeptide set forth in SEQ ID NO: 2 with at least one conservative amino acid substitution, wherein the encoded polypeptide has an activity of the polypeptide set forth in SEQ ID NO: 2;

(b) a nucleotide sequence encoding a polypeptide set forth in SEQ ID NO: 2 with at least one amino acid insertion, wherein the encoded polypeptide has an activity of the polypeptide set forth in SEQ ID NO: 2;

(c) a nucleotide sequence encoding a polypeptide set forth in SEQ ID NO: 2 with at least one amino acid deletion, wherein the encoded polypeptide has an activity of the polypeptide set forth in SEQ ID NO: 2;

(d) a nucleotide sequence encoding a polypeptide set forth in SEQ ID NO: 2 which has a C- and/or N-terminal truncation, wherein the encoded polypeptide has an activity of the polypeptide set forth in SEQ ID NO: 2;

(e) a nucleotide sequence encoding a polypeptide set forth in SEQ ID NO: 2 with at least one modification selected from the group consisting of amino acid substitutions, amino acid insertions, amino acid deletions, C-terminal truncation, and N-terminal truncation, wherein the polypeptide has an activity of the encoded polypeptide set forth in SEQ ID NO: 2;

(f) a nucleotide sequence of (a)-(e) comprising a fragment of at least about 16 nucleotides;

(g) a nucleotide sequence which hybridizes under moderately or highly stringent conditions to the complement of any of (a)-(f), wherein the encoded polypeptide has an activity of the polypeptide set forth in SEQ ID NO: 2; and

(h) a nucleotide sequence complementary to any of (a)-(e).

The invention also provides for an expression vector comprising the isolated nucleic acid molecules set forth herein; recombinant host cells (eukaryotic and/or prokaryotic) that comprise the vector; the process for producing a h2520 polypeptide comprising culturing the host cell under suitable conditions to express the polypeptide and optionally isolating the polypeptide from the culture; and the isolated polypeptide produced by this process. The nucleic acid molecule used in this process may also comprise promoter DNA other than the promoter DNA for the native Cdk11 polypeptide operatively linked to the nucleotide sequence encoding the Cdk11 polypeptide.

The invention also provides for a nucleic acid molecule as described in the previous paragraphs wherein the percent identity is determined using a computer program selected from the group consisting of GAP, BLASTP, BLASTN, FASTA, BLASTA, BLASTX, BestFit, and the Smith-Waterman algorithm.

The invention also provides for an isolated polypeptide comprising the amino acid sequence selected from the group consisting of:

(a) the mature amino acid sequence set forth in SEQ ID NO: 2 comprising a mature amino terminus at residue 1, and optionally further comprising an amino-terminal methionine;

(b) an amino acid sequence for an ortholog of SEQ ID NO: 2, wherein the polypeptide has an activity of the polypeptide set forth in SEQ ID NO: 2;

(c) an amino acid sequence that is at least about 70, 75, 80, 85, 90, 95, 96, 97, 98, or 99 percent identical to the amino acid sequence of SEQ ID NO: 2, wherein the polypeptide has an activity of the polypeptide set forth in SEQ ID NO: 2 as determined using a computer program selected from the group consisting of GAP, BLASTP, BLASTN, FASTA, BLASTA, BLASTX, BestFit, and the Smith-Waterman algorithm;

(d) a fragment of the amino acid sequence set forth in SEQ ID NO: 2 comprising at least about 25 amino acid residues, wherein the polypeptide has an activity of the polypeptide set forth in SEQ ID NO: 2; and

(e) an amino acid sequence for an allelic variant or splice variant of either the amino acid sequence set forth in SEQ ID NO: 2, or at least one of (a)-(c) wherein the polypeptide has an activity of the polypeptide set forth in SEQ ID NO: 2.

The invention further provides for an isolated polypeptide comprising the amino acid sequence selected from the group consisting of:

(a) the amino acid sequence set forth in SEQ ID NO: 2 with at least one conservative amino acid substitution, wherein the polypeptide has an activity of the polypeptide set forth in SEQ ID NO: 2;

(b) the amino acid sequence set forth in SEQ ID NO: 2 with at least one amino acid insertion, wherein the polypeptide has an activity of the polypeptide set forth in SEQ ID NO: 2;

(c) the amino acid sequence set forth in SEQ ID NO: 2 with at least one amino acid deletion, wherein the polypeptide has an activity of the polypeptide set forth in SEQ ID NO: 2;

(d) the amino acid sequence set forth in SEQ ID NO: 2 which has a C- and/or N-terminal truncation, wherein the polypeptide has an activity of the polypeptide set forth in SEQ ID NO: 2; and

(e) the amino acid sequence set forth in SEQ ID NO: 2, with at least one modification selected from the group consisting of amino acid substitutions, amino acid insertions, amino acid deletions, C-terminal truncation, and N-terminal truncation, wherein the polypeptide has an activity of the polypeptide set forth in SEQ ID NO: 2.

Also provided are fusion polypeptides comprising the polypeptide sequences of (a)-(e) above of the preceding paragraphs.

The present invention also provides for an expression vector comprising the isolated nucleic acid molecules set forth herein, recombinant host cells comprising recombinant nucleic acid molecules set forth herein, and a method of producing a Cdk11 polypeptide comprising culturing the host cells and optionally isolating the polypeptide so produced. These expression vectors include baculovirus expression vectors which utilize insect cells for expression.

A transgenic non-human animal comprising a nucleic acid molecule encoding a Cdk11 polypeptide is also encompassed by the invention. The Cdk11 nucleic acid molecules are introduced into the animal in a manner that allows expression and increased levels of the Cdk11 polypeptide, which may include increased circulating levels. The transgenic non-human animal is preferably a mammal. Also provided is a transgenic non-human animal comprising a disruption in the nucleic acid molecule encoding a Cdk11 polypeptide, which will knock-out or significantly decrease expression of the Cdk11 polypeptide.

Also provided are derivatives of the Cdk11 polypeptides of the present invention.

Analogs of Cdk11 are provided for in the present invention which result from conservative and non-conservative amino acids substitutions of the Cdk11 polypeptide of SEQ ID NO: 2. Such analogs include a Cdk11 polypeptide wherein the amino acid at position 48 is selected from the group consisting of valine, isoleucine, leucine, or alanine, a Cdk11 polypeptide wherein the amino acid at position 173 is selected from the group consisting of glutamic acid or aspartic acid, a Cdk11 polypeptide wherein the amino acid at position 172 is selected from the group consisting of proline or alanine, a Cdk11 polypeptide wherein the amino acid at position 169 is selected from the group consisting of tyrosine, tryptophan, or phenylalanine, a Cdk11 polypeptide wherein the amino acid at position 145 is selected from the group consisting of aspartic acid or glutamic acid, and a Cdk11 polypeptide wherein the amino acid at position 128 is selected from the group consisting of leucine, norleucine, isoleucine, valine, methionine, alanine or phenylalanine.

The present invention further provides for an antibody or fragment thereof that specifically binds an Cdk11 polypeptide as set forth herein. This antibody can be polyclonal or monoclonal, and can be produced by immunizing an animal with a peptide comprising an amino acid sequence of SEQ ID NO: 2.

Also provided is the hybridoma that produces a monoclonal antibody that binds to a peptide comprising an amino acid sequence of SEQ ID NO: 2.

The present invention also provides for a method of detecting or quantitating the amount of Cdk11 polypeptide in a sample comprising contacting a sample suspected of containing Cdk11 polypeptide with the anti-Cdk11 antibody or antibody fragment set forth herein and detecting the binding of said antibody or antibody fragment.

Additionally provided by the invention are selective binding agents or fragments thereof that are capable of specifically binding the Cdk11 polypeptides, derivatives, variants, and fragments (preferably having sequences of at least about 25 amino acids) thereof. These selective binding agents may be antibodies such as humanized antibodies, human antibodies, polyclonal antibodies, monoclonal antibodies, chimeric antibodies, complementarity determining region CDR)-grafted antibodies, anti-idiotypic antibodies, and fragments thereof. Furthermore, the selective binding agents may be antibody variable region fragments, such as Fab or Fab′ fragments, or fragments thereof, and may comprise at least one complementarity determining region with specificity for a Cdk11 polypeptide set forth herein. The selective binding agent may also be bound to a detectable label, such as a radiolabel, a fluorescent label, an enzyme label, or any other label known in the art. Further, the selective binding agent may antagonize Cdk 11 polypeptide biological activity, and/or be produced by immunizing an animal with a Cdk11 polypeptide as set forth herein.

The present invention also provides for a hybridoma that produces a selective binding agent capable of binding Cdk11 polypeptide as set forth herein.

Also provided is a method for treating, preventing, or ameliorating a disease, condition, or disorder comprising administering to a patient an effective amount of a selective binding agent as set forth herein. An effective amount, or a therapeutically effective amount, is an amount sufficient to result in a detectable change in the course or magnitude of the disease, condition or disorder, such as the intensity or duration of presentment of any symptom associated therewith.

Pharmaceutical compositions comprising the above-described nucleic acid molecules, polypeptides, or selective binding agents and one or more pharmaceutically acceptable formulation agents are also encompassed by the invention. The pharmaceutically acceptable formulation agent may be a carrier, adjuvant, solubilizer, stabilizer, or anti-oxidant. The nucleic acid molecules of the present invention may be contained in viral vectors. The pharmaceutical compositions are used to provide therapeutically effective amounts of the nucleic acid molecules or polypeptides of the present invention.

Also provided are derivatives of the Cdk11 polypeptides of the present invention. These polypeptides may be covalently modified with a water-soluble polymer wherein the water-soluble polymer is selected from the group consisting of polyethylene glycol, monomethoxy-polyethylene glycol, dextran, cellulose, poly-(N-vinyl pyrrolidone) polyethylene glycol, propylene glycol homopolymers, polypropylene oxide/ethylene oxide co-polymers, polyoxyethylated polyols, and polyvinyl alcohol.

The present invention also provides for fusion polypeptides comprising the polypeptide sequences set forth herein fused to a heterologous amino acid sequence, which may be an IgG constant domain or fragment thereof.

Pharmaceutical compositions comprising the nucleotides, polypeptides, or selective binding agents of the present invention and one or more pharmaceutically acceptable formulation agents are also encompassed by the invention. The pharmaceutical compositions are used to provide therapeutically effective amounts of the nucleotides or polypeptides of the present invention. The invention is also directed to methods of using the polypeptides, nucleic acid molecules, and selective binding agents. The invention also provides for devices to administer a Cdk11 polypeptide encapsulated in a membrane.

The Cdk11 polypeptides and nucleic acid molecules of the present invention may be used to treat, prevent, ameliorate, diagnose and/or detect diseases and disorders, including those recited herein. Expression analysis in biological, cellular or tissue samples suggests that Cdk11 polypeptide may play a role in the diagnosis and/or treatment of hyperproliferative diseases such as immune disorders, angiogenesis and vasculogenesis, wound healing, diabetes mellitus, psoriasis, liver disease, inflammation and cancer. This expression can de detected with a diagnostic agent such as a Cdk11 polynucleotide.

The invention encompasses diagnosing a pathological condition or a susceptibility to a pathological condition in a subject caused by or resulting from abnormal levels of Cdk11 polypeptide comprising determining the presence or amount of expression of the Cdk11 polypeptide in a sample; and comparing the level of said polypeptide in a biological, tissue or cellular sample from either normal subjects or the subject at an earlier time, wherein susceptibility to a pathological condition is based on the presence or amount of expression of the polypeptide.

The present invention also provides a method of assaying test molecules to identify a test molecule which binds to a Cdk11 polypeptide. The method comprises contacting a Cdk11 polypeptide with a test molecule and to determine the extent of binding of the test molecule to the polypeptide. The method further comprises determining whether such test molecules are agonists or antagonists of a Cdk11 polypeptide. The present invention further provides a method of testing the impact of molecules on the expression of Cdk11 polypeptide or on the activity of Cdk11 polypeptide.

The present invention provides for methods of identifying antagonists of Cdk11 biological activity comprising contacting a small molecule compound with Cdk11 polypeptides and measuring Cdk11 biological activity in the presence and absence of these small molecules. These small molecules can be a naturally occurring medicinal compound or derived from combinational chemical libraries. In addition, the present invention also encompasses methods which identify Cdk11 binding partners, such as cyclins. These methods utilize a yeast two-hybrid approach comprising a bait construct consisting of a Cdk11 polynucleotide fused to GAL4 DNA binding domain. The bait construct is used to screen a cDNA library, wherein the library consists of nucleotide sequences fused to a GALA activation domain. Library sequences encoding Cdk11 interacting proteins can be identified by the transcriptional activation of reporter genes under the control of GAL4. See Guarente, Trend Gen., 9: 342-346 (1993); Bartel & Field, Meth. Enz., 254: 241-63 (1995).

The present invention also encompasses methods of identifying substrates for Cdk11 kinase activity. These methods comprise a phage display peptide library, contacting this phage display library with a Cdk11 polypeptide and detecting changes in phosphorylation within the phage display library with an antibody that detects phosphorylation (phosph-specific antibodies) such as antibodies that specifically bind phospho-serine or phospho-threonine.

Methods of regulating expression and modulating i.e., increasing or decreasing) levels of a Cdk11 polypeptide are also encompassed by the invention. One method comprises administering to an animal a nucleic acid molecule encoding a Cdk11 polypeptide. In another method, a nucleic acid molecule comprising elements that regulate or modulate the expression of a Cdk11 polypeptide may be administered. Examples of these methods include gene therapy, cell therapy, and anti-sense therapy as further described herein.

In another aspect of the present invention, the Cdk11 polypeptides may be used for identifying binding partners thereof (“Cdk11 binding partners” such as cyclins and other Cdk11 cofactors). Yeast two-hybrid screens have been extensively used to identify and clone binding partners and receptors for proteins. (Chien et al., Proc. Natl. Acad. Sci. USA, 88:9578-9583, 1991) The isolation of a Cdk11 binding partner(s) is useful for identifying or developing novel agonists and antagonists of the Cdk11 activity.

Such agonists and antagonists include soluble Cdk11 cofactors (such as cyclins), Cdk inhibitors, anti-Cdk11 selective binding agents (such as Cdk 11 antibodies and derivatives thereof), small molecules, peptides or derivatives thereof capable of binding Cdk11 polypeptides, or antisense oligonucleotides, any of which can be used for potentially treating one or more diseases or disorders, including those recited herein.

In certain embodiments, a Cdk11 polypeptide agonist or antagonist may be a protein, peptide, carbohydrate, lipid, or small molecular weight molecule which interacts with Cdk11 polypeptide to regulate its activity.

The invention also provides for devices comprising a membrane suitable for implantation to administer a Cdk11 polypeptide, wherein Cdk11 polypeptide or cells which can secrete said peptide may be encapsulated in the membrane. The said membrane is permeable to the Cdk11 polypeptide; preferably, the membrane is impermeable to detrimental materials such as materials larger than the polypeptide.

A transgenic non-human animal comprising a nucleic acid molecule encoding a Cdk11 polypeptide is also encompassed by the invention. The Cdk11 nucleic acid molecule is introduced into the animal in a manner that allows expression and increased levels of the Cdk11 polypeptide, which may include increased circulating levels. The transgenic non-human animal is preferably a mammal.

The present invention provides for a diagnostic reagent comprising a detectably labeled polynucleotide encoding the amino acid sequence set out in SEQ ID NO: 2, or a fragment, variant or homolog thereof, including allelic variants and spliced variants thereof. The detectably labeled polynucleotide may be a first-strand cDNA, DNA, or RNA.

The invention also provides a method for detecting the presence of Cdk11 nucleic acid molecules in a biological sample comprising the steps of:

(a) providing a biological sample suspected of containing Cdk 11 nucleic acid molecules;

(b) contacting the biological sample with a diagnostic reagent under conditions wherein the diagnostic reagent will hybridize with Cdk11 nucleic acid molecules contained in said biological sample;

(c) detecting hybridization between Cdk11 nucleic acid molecules in the biological sample and the diagnostic reagent; and

(d) comparing the level of hybridization between the biological sample and diagnostic reagent with the level of hybridization between a known concentration of Cdk11 nucleic acid molecules and the diagnostic reagent.

The invention also provides a method for detecting the presence of Cdk 11 nucleic acid molecules in a tissue or cellular sample-comprising the steps of:

(a) providing a tissue or cellular sample suspected of containing Cdk11 nucleic acid molecules;

(b) contacting the tissue or cellular sample with a diagnostic reagent under conditions wherein the diagnostic reagent will hybridize with Cdk11 nucleic acid molecules;

(c) detecting hybridization between Cdk11 nucleic acid molecules in the tissue or cellular sample and the diagnostic reagent; and

(d) comparing the level of hybridization between the tissue or cellular sample and diagnostic reagent with the level of hybridization between a known concentration of Cdk11 nucleic acid molecules and the diagnostic reagent.

Also provided in the present invention is a polynucleotide described above attached to a solid support, as well as an array of polynucleotides comprising at least one polynucleotide as described above.

### BRIEF DESCRIPTION OF THE FIGURES

FIG. 1A-1E presents a nucleic acid sequence (SEQ ID NO: 1) which encodes the human Cdk11 polypeptide sequence (SEQ ID NO: 2).

FIG. 2 presents an alignment of the predicted amino acid sequence of Cdk11 polypeptide (SEQ ID NO: 2) with the polypeptide sequence of cyclin dependent kinase family members Cdk7, Cdk3, Cdk2, and Cdk1 (SEQ ID NOS: 16-19, respectively) using the Pileup Program (Wisconsin GCC Program Package ver. 8.1).

FIG. 3A-3F presents an alignment of the polynucleotide sequence of Cdk11 (SEQ ID NO: 1) with the published Genbank sequence polynucleotide sequence (accession no. AF035013; SEQ ID NO: 6) to display the errors in the published sequence.

FIG. 4 presents an alignment of the predicted amino acid sequence of Cdk11 polypeptide (SEQ ID NO: 2) with the predicted amino acid sequence of the published Genbank sequence (accession no. AF035013; SEQ ID NO: 7) to display the errors in the published sequence.

### DETAILED DESCRIPTION OF THE INVENTION

The section headings used herein are for organizational purposes only and are not to be construed as limiting the subject matter described therein. All references cited in this application are expressly incorporated by reference herein.

The term “Cdk11 nucleic acid molecule” or “polynucleotide” refers to a nucleic acid molecule comprising or consisting of a nucleotide sequence set forth in SEQ ID NO: 1, a nucleotide sequence encoding the polypeptide set forth in SEQ ID NO: 2 or a nucleotide sequence encoding the polypeptide set forth in SEQ ID NO: 2, or the nucleic acid sequence of the DNA insert in American Type Culture Collection (ATCC), 10801 University Boulevard, Manassas, Va. 20110-2209, deposit No. PTA-2649, deposited on Nov. 3, 2000.

The term “Cdk11 polypeptide” refers to a polypeptide comprising the amino acid sequence of SEQ ID NO: 2, and related polypeptides. Related polypeptides include: Cdk11 polypeptide allelic variants, Cdk11 polypeptide analogs, Cdk11 polypeptide orthologs, Cdk11 polypeptide splice variants, Cdk11 polypeptide variants and Cdk11 polypeptide derivatives. Cdk11 polypeptides may be mature polypeptides, as defined herein, and may or may not have an amino terminal methionine residue, depending on the method by which they are prepared.

The term “Cdk11 polypeptide allelic variant” refers to the polypeptide encoded by one of several possible naturally occurring alternate forms of a gene occupying a given locus on a chromosome of an organism or a population of organisms.

The term “Cdk11 polypeptide derivatives” refers to a polypeptide having the an amino acid sequence as set forth in SEQ ID NO: 2, Cdk11 polypeptide allelic variants, Cdk11 polypeptide orthologs, Cdk11 polypeptide splice variants, or Cdk11 polypeptide variants, as defined herein, that have been chemically modified.

The term “Cdk11 polypeptide fragment” refers to a polypeptide that comprises a truncation at the amino terminus (with or without a leader sequence) and/or a truncation at the carboxy terminus of the polypeptide set forth in SEQ ID NO: 2, Cdk 11 polypeptide allelic variants, Cdk11 polypeptide analogs, Cdk 11 polypeptide orthologs, Cdk11 polypeptide splice variants and/or a Cdk11 polypeptide variant having one or more amino acid additions or substitutions or internal deletions (wherein the resulting polypeptide is at least 6 amino acids or more in length) as compared to the Cdk11 polypeptide amino acid sequence set forth in SEQ ID NO: 2. Cdk11 polypeptide fragments may result from alternative RNA splicing or from in vivo protease activity. In preferred embodiments, truncations comprise about 10 amino acids, or about 20 amino acids, or about 50 amino acids, or about 75 amino acids, or about 100 amino acids, or more than about 100 amino acids. The polypeptide fragments so produced will comprise about 25 contiguous amino acids, or about 50 amino acids, or about 75 amino acids, or about 100 amino acids, or about 150 amino acids, or about 200 amino acids. Such Cdk11 polypeptide fragments may optionally comprise an amino terminal methionine residue. It will be appreciated that such fragments can be used, for example, to generate antibodies to Cdk11 polypeptides.

The term “Cdk11 fusion polypeptide” refers to a fusion of one or more amino acids (such as a heterologous peptide or polypeptide) at the amino or carboxy terminus of the polypeptide set forth in SEQ ID NO: 2, Cdk11 polypeptide allelic variants, Cdk11 polypeptide analogs, Cdk11 polypeptide orthologs, Cdk11 polypeptide splice variants, or Cdk 11 polypeptide variants having one or more amino acid deletions, substitutions or internal additions as compared to the Cdk11 polypeptide amino acid sequence set forth in SEQ ID NO: 2.

The term “Cdk11 polypeptide analog” refers to a related polypeptide with a similar amino acid sequence to the Cdk11 amino acid sequence set forth as SEQ ID NO: 2 with conserved and non-conserved amino acid substitutions.

The term “Cdk11 polypeptide ortholog” refers to a polypeptide from another species that corresponds to Cdk11 polypeptide amino acid sequence set forth in SEQ ID NO: 2. For example, mouse and human Cdk11 polypeptides are considered orthologs of each other.

The term “Cdk 11 polypeptide splice variant” refers to a nucleic acid molecule, usually RNA, which is generated by alternative processing of intron sequences in an RNA transcript containing the non-contiguous coding region of the Cdk11 polypeptide amino acid sequence set forth in SEQ ID NO: 2.

The term “Cdk11 polypeptide variants” refers to Cdk11 polypeptides comprising amino acid sequences having one or more amino acid sequence substitutions, deletions (such as internal deletions and/or Cdk11 polypeptide fragments), and/or additions (such as internal additions and/or Cdk11 fusion polypeptides) as compared to the Cdk11 polypeptide amino acid sequence set forth in SEQ ID NO: 2 (with or without a leader sequence). Variants may be naturally occurring (e.g., Cdk11 polypeptide allelic variants, Cdk11 polypeptide orthologs and Cdk11 polypeptide splice variants) or may be artificially constructed. Such Cdk11 polypeptide variants may be prepared from the corresponding nucleic acid molecules having a DNA sequence that varies accordingly from the DNA sequence set forth in SEQ ID NO: 1. In preferred embodiments, the variants have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, insertions, additions and/or deletions, wherein the substitutions may be conservative, or non-conservative, or any combination thereof.

The term “antigen” refers to a molecule or a portion of a molecule capable of being bound by a selective binding agent, such as an antibody, and additionally capable of being used in an animal to produce antibodies capable of binding to an epitope of each antigen. An antigen may have one or more epitopes.

The term “biologically active Cdk11 polypeptides” refers to Cdk11 polypeptides having at least one activity characteristic of the polypeptide comprising the amino acid sequence of SEQ ID NO: 2. An example of a potential Cdk11 biological activity is kinase activity, such as phosphorylation on serine or threonine residues on a substrate.

The term “phage display library” refers to a library of greater than 100 unique, random peptides of 7 to 30 amino acids in length. A phage display library can be screened to identify substrates, binding partners, and modulators of Cdk 11 biological activity.

The terms “effective amount” and “therapeutically effective amount” each refer to the amount of a Cdk11 polypeptide or Cdk11 nucleic acid molecule used to support an observable level of one or more biological activities of the Cdk11 polypeptides set forth herein.

The term “expression vector” refers to a vector which is suitable for use in a host cell and contains nucleic acid sequences which direct and/or control the expression of heterologous nucleic acid sequences. Expression includes, but is not limited to, processes such as transcription, translation, and RNA splicing, if introns are present.

The term “baculovirus” refers to a virus that consists of double-stranded, circular, super-coiled DNA molecules in rod-shaped capsids. These viruses infect insect populations and are commonly used to express heterologous genes in cultured insect cells and larvae.

The term “baculovirus expression vector” refers to an expression vector which is suitable for the transfer of a nucleotide sequence into the baculovirus genome (termed “Bacmid DNA”) by homologous recombination. The recombinant baculovirus particles produced upon transfection of the recombinant viral DNA into insect cells are subsequently used to infect insect cells resulting in high level, eukaryotic expression of the gene of interest.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “identity,” as known in the art, refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “similarity” is a related concept, but in contrast to “identity”, refers to a measure of similarity which includes both identical matches and conservative substitution matches. If two polypeptide sequences have, for example, 10/20 identical amino acids, and the remainder are all non-conservative substitutions, then the percent identity and similarity would both be 50%. If, in the same example, there are five more positions where there are conservative substitutions, then the percent identity remains 50%, but the percent similarity would be 75% (15/20). Therefore, in cases where there are conservative substitutions, the degree of percent similarity between two polypeptides will be higher than the percent identity between those two polypeptides.

The term “isolated nucleic acid molecule” refers to a nucleic acid molecule of the invention that (1) has been separated from at least about 50 percent of proteins, lipids, carbohydrates or other materials with which it is naturally found when total DNA is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

The term “isolated polypeptide” refers to a polypeptide of the present invention that (1) has been separated from at least about 50 percent of polynucleotides, lipids, carbohydrates or other materials with which it is naturally found when isolated from the source cell, (2) is not linked (by covalent or noncovalent interaction) to all or a portion of a polypeptide to which the “isolated polypeptide” is linked in nature, (3) is operably linked (by covalent or noncovalent interaction) to a polypeptide with which it is not linked in nature, or (4) does not occur in nature. Preferably, the isolated polypeptide is substantially free from any other contaminating polypeptides or other contaminants that are found in its natural environment that would interfere with its therapeutic, diagnostic, prophylactic or research use.

The term “mature Cdk11 polypeptide” refers to a Cdk11 polypeptide lacking a leader sequence. A mature Cdk11 polypeptide may also include other modifications such as proteolytic processing of the amino terminus (with or without a leader sequence) and/or the carboxy terminus, cleavage of a smaller polypeptide from a larger precursor, N-linked and/or O-linked glycosylation, and the like. An exemplary mature Cdk11 polypeptide is depicted by amino acid residue 1 through amino acid residue 346 of SEQ ID NO: 2.

The terms “nucleic acid sequence” or “nucleic acid molecule” refer to a DNA or RNA sequence. The terms encompass molecules formed from any of the known base analogs of DNA and RNA such as, but not limited to 4-acetylcytosine, 8-hydroxy-N6-methyladenine, aziridinyl-cytosine, pseudoisocytosine, 5-(carboxyhydroxylmethyl)uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5′-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, 2-thiocytosine, and 2,6-diaminopurine.

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” as used herein refers to a material that is not found in nature or that has been structurally modified or synthesized by man.

The term “operably linked” is used herein to refer to a method of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably linked” to the coding sequence.

The terms “pharmaceutically acceptable carrier” or “physiologically acceptable carrier” as used herein refer to one or more formulation materials suitable for accomplishing or enhancing the delivery of the Cdk11 polypeptide, Cdk11 nucleic acids molecule, or Cdk11 selective binding agent as a pharmaceutical composition.

The term “selective binding agent” refers to a molecule or molecules having specificity for a Cdk11 polypeptide. As used herein the terms “specific” and “specifically” refer to the ability of the selective binding agents to bind to human Cdk11 polypeptides and not to bind to human non-Cdk11 polypeptides. It will be appreciated, however, that the selective binding agents may also bind orthologs of the polypeptide set forth in SEQ ID NO: 2, that is, interspecies versions thereof, such as mouse and rat polypeptides.

The term “phospho-specific antibody” refers to an antibody which specifically binds to phosphorylated amino acids, such as a phosphorylated tyrosine residue, phosphorylated threonine residue or a phosphorylated serine residue.

The term “transduction” is used to refer to the transfer of nucleic acid from one bacterium to another, usually by a phage. “Transduction” also refers to the acquisition and transfer of eukaryotic cellular sequences by viruses such as retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52: 456, 1973; Sambrook et al., Molecular Cloning, A Laboratory Manual, Cold Spring Harbor Laboratories (New York, 1989); Davis et al., Basic Methods in Molecular Biology, Elsevier, 1986; and Chu et al., Gene, 13:197, 1981. Such techniques can be used to introduce one or more exogenous DNA moieties into suitable host cells.

The term “transformation” as used herein refers to a change in a cell's genetic characteristics, and a cell has been transformed when it has been modified to contain new DNA. For example, a cell is transformed where it is genetically modified from its native state. Following transfection or transduction, the transforming DNA may recombine with that of the cell by physically integrating into a chromosome of the cell, it may be maintained transiently as an episomal element without being replicated, or it may replicate independently as a plasmid. A cell is considered to have been stably transformed when the DNA is replicated with the division of the cell.

The term “vector” is used to refer to any molecule (e.g., nucleic acid, plasmid, or virus) used to transfer coding information to a host cell.

Relatedness of Nucleic Acid Molecules and/or Polypeptides

It is understood that related nucleic acid molecules include allelic or splice variants of the nucleic acid molecule of SEQ ID NO: 1, and include sequences which are complementary to any of the above nucleotide sequences. Related nucleic acid molecules also include a nucleotide sequence encoding a polypeptide comprising or consisting essentially of a substitution, modification, addition and/or deletion of one or more amino acid residues compared to the polypeptide in SEQ ID NO: 2.

Fragments include molecules which encode a polypeptide of at least about 25 amino acid residues, or about 50, or about 75, or about 100, or greater than about 100 amino acid residues of the polypeptide of SEQ ID NO: 2.

In addition, related Cdk11 nucleic acid molecules include those molecules which comprise nucleotide sequences which hybridize under moderately or highly stringent conditions as defined herein with the fully complementary sequence of the nucleic acid molecule of SEQ ID NO: 1, or of a molecule encoding a polypeptide, which polypeptide comprises the amino acid sequence as shown in SEQ ID NO: 2, or of a nucleic acid fragment as defined herein, or of a nucleic acid fragment encoding a polypeptide as defined herein. Hybridization probes may be prepared using the Cdk11 sequences provided herein to screen cDNA, genomic or synthetic DNA libraries for related sequences. Regions of the DNA and/or amino acid sequence of Cdk11 polypeptide that exhibit significant identity to known sequences are readily determined using sequence alignment algorithms as described herein and those regions may be used to design probes for screening.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual, 2^{nd} Ed., Cold Spring Harbor Laboratory, (Cold Spring Harbor, N.Y. (1989) and Anderson et al., Nucleic Acid Hybridization: a Practical Approach, Ch. 4, IRL Press Limited (Oxford, England).

More stringent conditions (such as higher temperature, lower ionic strength, higher formamide, or other denaturing agent) may also be used; however, the degree of hybridization will be affected. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate (NaDodSO₄ or SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4; however, at typical ionic strength conditions, the rate of hybridization is nearly independent of pH. (See Anderson et al., Nucleic Acid Hybridization: a Practical Approach, Ch. 4, IRL Press Limited (Oxford, England)).

Factors affecting the stability of DNA duplex include base composition, length, and degree of base pair mismatch. Hybridization conditions can be adjusted by one skilled in the art in order to accommodate these variables and allow DNAs of different sequence relatedness to form hybrids. The melting temperature of a perfectly matched DNA duplex can be estimated by the following equation:
Tₘ(° C.)=81.5+16.6(log [Na+])+0.41(% G+C)−600/N−0.72(% formamide)
where N is the length of the duplex formed in nucleotides, [Na⁺] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, a “moderately stringent” condition of 50° C. in 0.015 M sodium ion will allow about a 21% mismatch.

It will be appreciated by those skilled in the art that there is no absolute distinction between “highly” and “moderately” stringent conditions. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, one skilled in the art can simply lower the temperature or raise the ionic strength.

A good estimate of the melting temperature in 1.0 M NaCl* for oligonucleotide probes up to about 20 nucleotides is given by:
Tm=2° C. per A-T base pair+4° C. per G-C base pair

*The sodium ion concentration in 6× salt sodium citrate (SSC) is 1.0 M. See Suggs et al., Developmental Biology Using Purified Genes, p. 683, Brown and Fox (eds.) (1981).

High stringency washing conditions for oligonucleotides are usually at a temperature of 0-5° C. below the Tm of the oligonucleotide in 6×SSC, 0.1% SDS for longer oligonucleotides.

In another embodiment, related nucleic acid molecules comprise or consist of a nucleotide sequence that is about 70 percent (70%) identical to the nucleotide sequence as shown in SEQ ID NO: 1, or comprise or consist essentially of a nucleotide sequence encoding a polypeptide that is about 70 percent (70%) identical to the polypeptide set forth in SEQ ID NO: 2. In preferred embodiments, the nucleotide sequences are about 75 percent, or about 80 percent, or about 85 percent, or about 90 percent, or about 95, 96, 97, 98, or 99 percent identical to the nucleotide sequence as shown in SEQ ID NO: 1, or the nucleotide sequences encode a polypeptide that is about 75 percent, or about 80 percent, or about 85 percent, or about 90 percent, or about 95, 96, 97, 98, or 99 percent identical to the polypeptide sequence set forth in SEQ ID NO: 2.

Differences in the nucleic acid sequence may result in conservative and/or non-conservative modifications of the amino acid sequence relative to the amino acid sequence of SEQ ID NO: 2.

Conservative modifications to the amino acid sequence of SEQ ID NO: 2 (and the corresponding modifications to the encoding nucleotides) will produce Cdk11 polypeptides having functional and chemical characteristics similar to those of a naturally occurring Cdk11 polypeptide. In contrast, substantial modifications in the functional and/or chemical characteristics of Cdk11 polypeptides may be accomplished by selecting substitutions in the amino acid sequence of SEQ. ID NO: 2 that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described Cunningham and Wells, Science 244: 1081-1085, 1989 for “alanine scanning mutagenesis.”

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties.

Naturally occurring residues may be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: H is, Lys, Arg;

5) residues that influence chain orientation: Gly, Pro; and

6) aromatic: Trp, Tyr, Phe.

For example, non-conservative substitutions may involve the exchange of a member of one of these classes for a member from another class. Such substituted residues may be introduced into regions of the human Cdk11 polypeptide that are homologous, or similar, with non-human Cdk11 polypeptide orthologs, or into the non-homologous regions of the molecule.

In making such changes, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157: 105-131, 1982. It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.”

Desired amino acid substitutions (whether conservative or non-conservative) can be determined by those skilled in the art at the time such substitutions are desired. For example, amino acid substitutions can be used to identify important residues of the Cdk11 polypeptide, or to increase or decrease the affinity of the Cdk11 polypeptides for their substrates described herein.

Exemplary amino acid substitutions are set forth in Table I.

TABLE 1
Conservative Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, | Leu |
| | Ala, Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of the polypeptide set forth in SEQ ID NO: 2 using well known techniques. For identifying suitable areas of the molecule that may be changed without destroying activity, one skilled in the art may target areas not likely to be important for activity. For example, when similar polypeptides with similar activities from the same species or from other species are known, one skilled in the art may compare the amino acid sequence of a Cdk11 polypeptide to such similar polypeptides. With such a comparison, one can identify residues and portions of the molecules that are conserved among similar polypeptides. It will be appreciated that changes in areas of a Cdk11 polypeptide that are not conserved relative to such similar polypeptides would be less likely to adversely affect the biological activity and/or structure of the Cdk11 polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a Cdk11 polypeptide that correspond to amino acid residues which are important for activity or structure in similar polypeptides. One skilled in the art may opt for chemically similar amino acid substitutions for such predicted important amino acid residues of Cdk11 polypeptides.

One skilled in the art can also analyze the three-dimensional structure and amino acid sequence in relation to that structure in similar polypeptides. In view of such information, one skilled in the art may predict the alignment of amino acid residues of a Cdk11 polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays know to those skilled in the art. Such variants could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

Cdk11 polypeptide analogs of the invention can be determined by comparing the amino acid sequence of Cdk11 polypeptide with related family members. Exemplary Cdk11 polypeptide related family members include, but are not limited to, Cdk1, Cdk2, Cdk3, Cdk5, and Cdk7. This comparison can be accomplished by using a Pileup alignment (Wisconsin GCG Program Package) or an equivalent (overlapping) comparison with multiple family members within conserved and non-conserved regions.

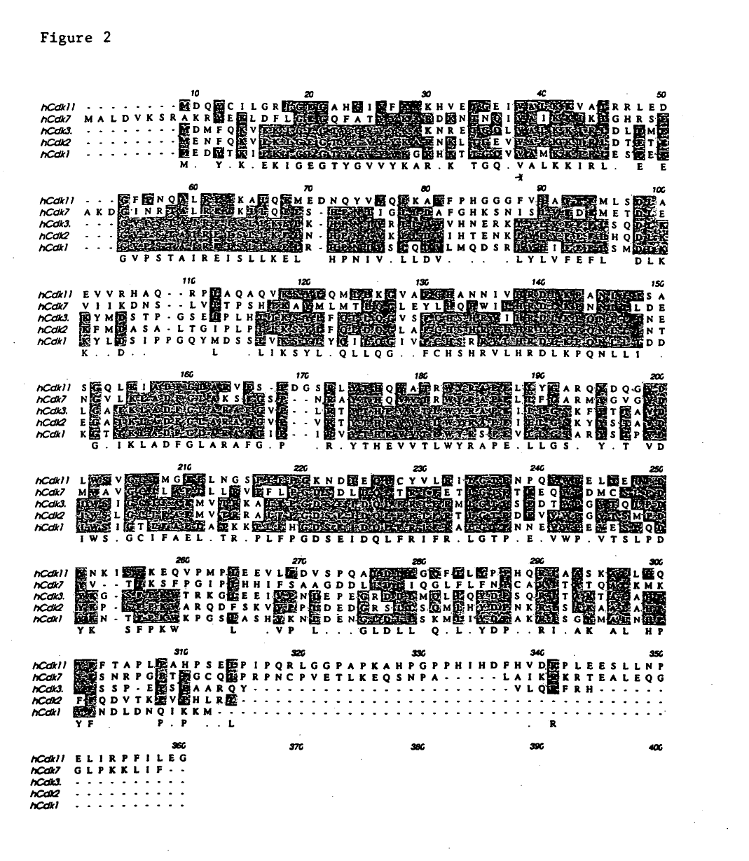

As shown in FIG. 2, the predicted amino acid sequence of Cdk11 polypeptide (SEQ ID NO: 2) is aligned with human Cdk7, human Cdk3, human Cdk2 and human Cdk1 (SEQ ID NOS: 16-19). Other Cdk11 polypeptide analogs can be determined using these or other methods known to those of skill in the art. These overlapping sequences provide guidance for conservative and non-conservative amino acids substitutions resulting in additional Cdk11 analogs. It will be appreciated that these amino acid substitutions can consist of naturally occurring or non-naturally occurring amino acids. For example, as depicted in FIG. 2, alignment of the of these cyclin dependent kinases indicates potential Cdk 11 analogs may have the Ala residue at position 48 of SEQ ID NO: 2 (position 59 on FIG. 2) substituted with a Val, Leu, or Ile residue, the Glu residue at position 173 of SEQ ID NO: 2 (position 187 on FIG. 2) may be substituted with an Asp residue, or the Pro residue at position 172 of SEQ ID NO: 2 (position 186 on FIG. 2) may be substituted with a Ala residue. Further, the Tyr residue at position 169 of SEQ ID NO: 2 (position 182 on FIG. 2) may be substituted with Trp or Phe, the Asp residue at position 145 of SEQ ID NO: 2 (position 158 on FIG. 2) may be substituted with Glu, and the Leu at position 128 of SEQ ID NO: 2 (position 141 on FIG. 2) may be substituted with Ile, Ala, Met, Phe, or norleucine.

A number of scientific publications have been devoted to the prediction of secondary structure. See Chou et al., Biochemistry, 13(2): 222-245, 1974; Chou et al., Biochemistry, 113(2): 211-222, 1974; Chou et al., Adv. Enzymol. Relat. Areas Mol. Biol., 47:45-148, 1978; Chou et al., Ann. Rev. Biochem., 47:251-276 and Chou et al., Biophys. J, 26: 367-384, 1979. Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1):244-247, 1999). It has been suggested (Brenner et al., Curr. Opin. Struct. Biol., 7(3):369-376, 1997) that there are a limited number of folds in a given polypeptide or protein and that once a critical number of structures have been resolved, structural prediction will become dramatically more accurate.

Additional methods of predicting secondary structure include “threading” (Jones et al., Current Opin. Struct. Biol., 7(3):377-87, 1997; Sippl et al., Structure, 4(1):15-9, 1996), “profile analysis” (Bowie et al., Science, 253:164-170, 1991; Gribskov et al., Meth. Enzym., 183:146-159, 1990; Gribskov et al., Proc. Nat. Acad. Sci., 84(13):4355-4358, 1987), and “evolutionary linkage” (See Home, supra, and Brenner, supra 1997).

In addition, the polypeptide comprising the amino acid sequence of SEQ ID NO: 2 or a Cdk 11 polypeptide variant may be fused to a homologous polypeptide to form a homodimer or to a heterologous polypeptide to form a heterodimer. Heterologous peptides and polypeptides include, but are not limited to: an epitope to allow for the detection and/or isolation of a Cdk11 fusion polypeptide; a transmembrane receptor protein or a portion thereof, such as an extracellular domain, or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; and a polypeptide which has a therapeutic activity different from the polypeptide comprising the amino acid sequence set forth in SEQ ID NO: 2 or a Cdk11 polypeptide variant.

Fusions can be made either at the amino terminus or at the carboxy terminus of the polypeptide comprising the amino acid sequence set forth in SEQ ID NO: 2 or a Cdk11 polypeptide variant. Fusions may be direct with no linker or adapter molecule or indirect using a linker or adapter molecule. A linker or adapter molecule may be one or more amino acid residues, typically from 20 to about 50 amino acid residues. A linker or adapter molecule may also be designed with a cleavage site for a DNA restriction endonuclease in an encoding polynucleotide or for a protease to allow for the separation of the fused moieties. It will be appreciated that once constructed, the fusion polypeptides can be derivatized according to the methods described herein.

In a further embodiment of the invention, the polypeptide comprising the amino acid sequence of SEQ ID NO: 2 or a Cdk11 polypeptide variant is fused to one or more domains of an Fc region of human IgG. Antibodies comprise two functionally independent parts, a variable domain known as “Fab”, which binds antigens, and a constant domain known as “Fc”, which is involved in effector functions such as complement activation and attack by phagocytic cells. An Fc has a long serum half-life, whereas an Fab is short-lived. Capon et al., Nature, 337: 525-31, 1989. When constructed together with a therapeutic protein, an Fc domain can provide longer half-life or incorporate such functions as Fc receptor binding, protein A binding, complement fixation and perhaps even placental transfer. Id. Table II summarizes the use of certain Fc fusions known in the art.

TABLE II
Fc Fusion with Therapeutic Proteins

| Form of | Fusion | Therapeutic | |
|---|---|---|---|
| Fc | partner | implications | Reference |
| IgG1 | N-terminus | Hodgkin's disease; | U.S. Pat. No. |
| | of CD30-L | anaplastic lymphoma; T- | 5,480,981 |
| | | cell leukemia | |
| Murine | IL-10 | anti-inflammatory; | Zheng et al., J. |
| Fcγ2a | | transplant rejection | Immunol., 154: |
| IgG1 | TNF receptor | septic shock | 5590-600, 1995 |
| | | | Fisher et al., N. |
| | | | Engl. J. Med., |
| | | | 334: 1697-1702, |
| | | | 1996; Van Zee |
| | | | et al., , J. |
| | | | Immunol., 156: |
| | | | 2221-30, 1996 |
| IgG, IgA, | TNF receptor | inflammation, | U.S. Pat. No. |
| IgM, or | | autoimmune disorders | 5,808,029, issued |
| IgE | | | Sep. 15, 1998 |
| (excluding | | | |
| the first | | | |
| domain) | | | |
| IgG1 | CD4 receptor | AIDS | Capon et al., |
| | | | Nature 337: 525- |
| | | | 31, 1989 |
| IgG1, | N-terminus | anti-cancer, antiviral | Harvill a al., |
| IgG3 | of IL-2 | | Immunotech., 1: |
| | | | 95-105, 1995 |
| IgG1 | C-terminus | osteoarthritis; | WO 97/23614, |
| | of OPG | bone density | published |
| | | | Jul. 3, 1997 |
| IgG1 | N-terminus | anti-obesity | PCT/US97/ |
| | of leptin | | 23183, filed |
| | | | Dec. 11, 1997 |
| Human Ig | CTLA-4 | autoimmune disorders | Linsley, J. |
| Cγl | | | Exp. Med., |
| | | | 174: 561-9, 1991 |

In one example, all or a portion of the human IgG hinge, CH2 and CH3 regions may be fused at either the N-terminus or C-terminus of the Cdk11 polypeptides using methods known to the skilled artisan. The resulting Cdk11 fusion polypeptide may be purified by use of a Protein A affinity column. Peptides and proteins fused to an Fc region have been found to exhibit a substantially greater half-life in vivo than the unfused counterpart. Also, a fusion to an Fc region allows for dimerization/multimerization of the Fusion polypeptide. The Fc region may be a naturally occurring Fc region, or may be altered to improve certain qualities, such as therapeutic qualities, circulation time, reduce aggregation, etc.

Identity and similarity of related nucleic acid molecules and polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J., Applied Math., 48:1073, 1988.

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12:387, 1984; Genetics Computer Group, University of Wisconsin, Madison, Wis.), BLASTP, BLASTN, and FASTA (Altschul et al., J. Mol. Biol., 215:403-410, 1990). The BLASTX program is publicly available from the National Center for Biotechnology Information (NCBI) and other sources (BLAST Manual, Altschul et al. NCB/NLM/NIH Bethesda, Md. 20894; Altschul et al, supra). The well-known Smith-Waterman algorithm may also be used to determine identity.

Certain alignment schemes for aligning two amino acid sequences may result in the matching of only a short region of the two sequences, and this small aligned region may have very high sequence identity even though there is no significant relationship between the two full-length sequences. Accordingly, in a preferred embodiment, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3, 1978 for the PAM 250 comparison matrix; Henikoff et al., Proc. Natl. Acad. Sci. USA, 89:10915-10919, 1992 for the BLOSUM 62 comparison matrix) is also used by the algorithm.

Preferred parameters for a polypeptide sequence comparison include the following:
Algorithm: Needleman et al., J. Mol. Biol., 48,443-453, 1970;
Comparison matrix: BLOSUM 62 from Henikoff et al., Proc. Natl. Acad. Sci. USA, 89: 10915-10919, 1992);
Gap Penalty: 12
Gap Length Penalty: 4
Threshold of Similarity: 0

The GAP program is useful with the above parameters. The aforementioned parameters are the default parameters for polypeptide comparisons (along with no penalty for end gaps) using the GAP algorithm.

Preferred parameters for nucleic acid molecule sequence comparisons include the following:

Algorithm: Needleman et al., J. Mol. Biol., 48: 443-453, 1970;

Comparison matrix: matches=+10, mismatch=0

Gap Penalty: 50

Gap Length Penalty: 3

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used by those of skill in the art, including those set forth in the Program Manual, Wisconsin Package, Version 9, September, 1997. The particular choices to be made will be apparent to those of skill in the art and will depend on the specific comparison to be made, such as DNA-to-DNA, protein-to-protein, protein-to-DNA; and additionally, whether the comparison is between given pairs of sequences (in which case GAP or BestFit are generally preferred) or between one sequence and a large database of sequences (in which case FASTA or BLASTA are preferred).

Synthesis

It will be appreciated by those skilled in the art that the nucleic acid and polypeptide molecules described herein may be produced by recombinant and other means.

Nucleic Acid Molecules

The nucleic acid molecules encode a polypeptide comprising the amino acid sequence of a Cdk11 polypeptide and can readily be obtained in a variety of ways including, without limitation, chemical synthesis, cDNA or genomic library screening, expression library screening and/or PCR amplification of cDNA.

Recombinant DNA methods used herein are generally those set forth in Sambrook et al., Molecular Cloning: A Laboratory Manual, Cold Spring Harbor Laboratory Press, Cold Spring Harbor, N.Y. (1989), and/or Ausubel et al., eds., Current Protocols in Molecular Biology, Green Publishers Inc. and Wiley and Sons, NY (1994). The present invention provides for nucleic acid molecules as described herein and methods for obtaining such molecules.

Where a gene encoding the amino acid sequence of a Cdk11 polypeptide has been identified from one species, all or a portion of that gene may be used as a probe to identify orthologs or related genes from the same species. The probes or primers may be used to screen cDNA libraries from various tissue sources believed to express the Cdk11 polypeptide. In addition, part or all of a nucleic acid molecule having the sequence set forth in SEQ ID NO: 1 may be used to screen a genomic library to identify and isolate a gene encoding the amino acid sequence of a Cdk11 polypeptide. Typically, conditions of moderate or high stringency will be employed for screening to minimize the number of false positives obtained from the screening.

Nucleic acid molecules encoding the amino acid sequence of Cdk11 polypeptides may also be identified by expression cloning which employs the detection of positive clones based upon a property of the expressed protein. Typically, nucleic acid libraries are screened by the binding of an antibody or other binding partner (e.g., receptor, ligand, or co-factor) to cloned proteins which are expressed and displayed on a host cell surface. The antibody or binding partner is modified with a detectable label to identify those cells expressing the desired clone.

Recombinant expression techniques conducted in accordance with the descriptions set forth below may be followed to produce these polynucleotides and to express the encoded polypeptides. For example, by inserting a nucleic acid sequence which encodes the amino acid sequence of a Cdk11 polypeptide into an appropriate vector, one skilled in the art can readily produce large quantities of the desired nucleotide sequence. The sequences can then be used to generate detection probes or amplification primers. Alternatively, a polynucleotide encoding the amino acid sequence of a Cdk11 polypeptide can be inserted into an expression vector. By introducing the expression vector into an appropriate host, the encoded Cdk11 polypeptide may be produced in large amounts.

Another method for obtaining a suitable nucleic acid sequence is the polymerase chain reaction (PCR). In this method, cDNA is prepared from poly(A)+RNA or total RNA using the enzyme reverse transcriptase. Two oligonucleotide primers, typically complementary to two separate regions of cDNA (oligonucleotides) encoding the amino acid sequence of a Cdk11 polypeptide, are then added to the cDNA along with a polymerase such as Taq polymerase, and the polymerase amplifies the cDNA region between the two primers.

Another means of preparing a nucleic acid molecule encoding the amino acid sequence of a Cdk11 polypeptide is chemical synthesis using methods well known to the skilled artisan such as those described by Engels et al., (Angew. Chem. Intl. Ed., 28: 716-734, 1989). These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the amino acid sequence of a Cdk11 polypeptide will be several hundred nucleotides in length. Nucleic acids larger than about 100 nucleotides can be synthesized as several fragments using these methods. The fragments can then be ligated together to form the full-length nucleotide sequence of a Cdk 11 polypeptide. Usually, the DNA fragment encoding the amino terminus of the polypeptide will have an ATG, which encodes a methionine residue. This methionine may or may not be present on the mature form of the Cdk11 polypeptide, depending on whether the polypeptide produced in the host cell is designed to be secreted from that cell. Other methods known to the skilled artisan may be used as well.

In certain embodiments, nucleic acid variants contain codons which have been altered for the optimal expression of a Cdk11 polypeptide in a given host cell. Particular codon alterations will depend upon the Cdk11 polypeptide(s) and host cell(s) selected for expression. Such “codon optimization” can be carried out by a variety of methods, for example, by selecting codons which are preferred for use in highly expressed genes in a given host cell. Computer algorithms which incorporate codon frequency tables such as “Ecohigh.cod” for codon preference of highly expressed bacterial genes may be used and are provided by the University of Wisconsin Package Version 9.0, Genetics Computer Group, Madison, Wis. Other useful codon frequency tables include “Celegans_high.cod”, “Celegans_low.cod”, “Drosophila_high.cod”, “Human_high.cod”, “Maize_high.cod”, and “Yeast_high.cod”.

Vectors and Host Cells

A nucleic acid molecule encoding the amino acid sequences of Cdk11 polypeptide may be inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of Cdk11 polypeptide may be amplified/expressed in prokaryotic, yeast, insect (baculovirus systems), and/or eukaryotic host cells. Selection of the host cell will depend in part on whether a Cdk11 polypeptide is to be post-translationally modified (e.g., glycosylated and/or phosphorylated). If so, yeast, insect, or mammalian host cells are preferable. For a review of expression vectors, see Meth. Enz., v. 185, D. V. Goeddel, ed. Academic Press Inc., San Diego, Calif. (1990).

Typically, expression vectors used in any of the host cells will contain sequences for plasmid maintenance and for cloning and expression of exogenous nucleotide sequences. Such sequences, collectively referred to as “flanking sequences” in certain embodiments, will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the Cdk11 polypeptide coding sequence; the oligonucleotide sequence encodes polyHis (such as hexahis), or another “tag” such as FLAG, HA (hemaglutinin influenza virus) or myc for which commercially available antibodies exist. This tag is typically fused to the polypeptide upon expression of the polypeptide, and can serve as a means for affinity purification of the Cdk11 polypeptide from the host cell. Affinity purification can be accomplished, for example, by column chromatography using antibodies against the tag as an affinity matrix or metal affinity chromatography (such as nickel columns with an affinity to 6his tags). Optionally, the tag can subsequently be removed from the purified Cdk11 polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), or synthetic, or the flanking sequences may be native sequences which normally function to regulate Cdk 11 polypeptide expression. As such, the source of a flanking sequence may be any prokaryotic or eukaryotic organism, any vertebrate or invertebrate organism, or any plant, provided that the flanking sequences is in and can be activated by, the host cell machinery.

The flanking sequences useful in the vectors of this invention may be obtained by any of several methods well known in the art. Typically, flanking sequences useful herein other than the endogenous Cdk11 gene flanking sequences will have been previously identified by mapping and/or by restriction endonuclease digestion and can thus be isolated from the proper tissue source using the appropriate restriction endonucleases. In some cases, the full nucleotide sequence of one or more flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with suitable oligonucleotide and/or flanking sequence fragments from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of the Cdk11 polypeptide. If the vector of choice does not contain an origin of replication site, one may be chemically synthesized based on a known sequence, and ligated into the vector. For example, the origin of replication from the plasmid pBR322 (Product No. 303-3s, New England Biolabs, Beverly, Mass.) is suitable for most gram-negative bacteria and various origins (e.g., SV40, polyoma, adenovirus, vesicular stomatitus virus (VSV) or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene which will be expressed. Amplification is the process wherein genes which are in greater demand for the production of a protein critical for growth are reiterated in tandem within the chromosome(s) of successive generations of recombinant cells. Examples of suitable selectable markers for mammalian cells include dihydrofolate reductase (DHFR) and thymidine kinase. The mammalian cell transformants are placed under selection pressure which only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes Cdk11 polypeptide. As a result, increased quantities of Cdk11 polypeptide are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the Cdk11 polypeptide to be expressed. The Shine-Dalgarno sequence is varied but is typically a polypurine (i.e., having a high A-G content). Many Shine-Dalgarno sequences have been identified, each of which can be readily synthesized using methods set forth herein and used in a prokaryotic vector.

A leader, or signal, sequence may be used to direct a Cdk11 polypeptide out of the host cell. Typically, a nucleotide sequence encoding the signal sequence is positioned in the coding region of the Cdk11 nucleic acid molecule, or directly at the 5′ end of the Cdk11 polypeptide coding region. Many signal sequences have been identified, and any of those that are functional in the selected host cell may be used in conjunction with the Cdk11 nucleic acid molecule. Therefore, a signal sequence may be homologous (naturally occurring) or heterologous to the Cdk11 gene or cDNA. Additionally, a signal sequence may be chemically synthesized using methods described herein. In most cases, the secretion of a Cdk11 polypeptide from the host cell via the presence of a signal peptide will result in the removal of the signal peptide from the secreted Cdk11 polypeptide. The signal sequence may be a component of the vector, or it may be a part of a Cdk11 nucleic acid molecule that is inserted into the vector.

Included within the scope of this invention is the use of either a nucleotide sequence encoding a native Cdk11 polypeptide signal sequence joined to a Cdk11 polypeptide coding region or a nucleotide sequence encoding a heterologous signal sequence joined to a Cdk11 polypeptide coding region. The heterologous signal sequence selected should be one that is recognized and processed, i.e., cleaved by a signal peptidase, by the host cell. For prokaryotic host cells that do not recognize and process the native Cdk11 signal sequence, the signal sequence is substituted by a prokaryotic signal sequence selected, for example, from the group of the alkaline phosphatase, penicillinase, or heat-stable enterotoxin B leaders. For yeast secretion, e native Cdk11 polypeptide signal sequence may be substituted by the yeast invertase, alpha factor, or acid phosphatase leaders. In mammalian cell expression the native signal sequence is satisfactory, although other mammalian signal sequences may be used.

In some cases, such as where glycosylation is desired in a eukaryotic host cell expression system, one may manipulate the various presequences to improve glycosylation or yield. For example, one may alter the peptidase cleavage e site of a particular signal peptide, or add presequences, which also may affect glycosylation. The final protein product may have, in the −1 position (relative to the first amino acid of the mature protein), one or more additional amino acids incident to expression, which may not have been totally removed. For example, the final protein product may have one or two amino acid residues found in the peptidase cleavage site, attached to the N-terminus. Alternatively, use of some enzyme cleavage sites may result in a slightly truncated form of the desired Cdk11 polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the Cdk11 gene, especially where the gene used is a full length genomic sequence or a fragment thereof. Where the intron is not naturally occurring within the gene (as for most cDNAs), the intron(s) may be obtained from another source. The position of the intron with respect to flanking sequences and the Cdk11 gene is generally important, as the intron must be transcribed to be effective. Thus, when a Cdk11 cDNA molecule is being transcribed, the preferred position for the intron is 3′ to the transcription start site, and 5′ to the polyA transcription termination sequence. Preferably, the intron or introns will be located on one side or the other (i.e., 5′ or 3′) of the cDNA such that it does not interrupt the coding sequence. Any intron from any source, including viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector.

The expression and cloning vectors of the present invention will typically contain a promoter that is recognized by the host organism and operably linked to the molecule encoding a Cdk11 polypeptide. Promoters are untranscribed sequences located upstream (5′) to the start codon of a structural gene (generally within about 100 to 1000 bp) that control the transcription of the structural gene. Promoters are conventionally grouped into one of two classes, inducible promoters and constitutive promoters. In this context, inducible promoters include repressible/depressible promoters and conventional inducible promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding a Cdk11 polypeptide by removing the promoter from the source DNA by restriction enzyme digestion and inserting the desired promoter sequence into the vector.

The native Cdk11 promoter sequence may be used to direct amplification and/or expression of Cdk11 nucleic acid molecule. A heterologous promoter is preferred, however, if it permits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any useful restriction sites.

Suitable promoters for use with yeast hosts are also well known in the art.

Yeast enhancers are advantageously used with yeast promoters. Suitable promoters for use with mammalian host cells are well known and include, but are not limited to, those obtained from the genomes of viruses such as polyoma virus, fowl pox virus, adenovirus (such as Adenovirus 2), bovine papilloma virus, avian sarcoma virus, cytomegalovirus (CMV), a retrovirus, hepatitis-B virus and most preferably Simian Virus 40 SV40). Other suitable mammalian promoters include heterologous mammalian promoters, e.g., heat-shock promoters and the actin promoter.

Additional promoters which may be of interest in controlling Cdk11 gene transcription include, but are not limited to: the SV40 early promoter region (Bernoist and Chambon, Nature, 290:304-310, 1981); the CMV promoter; the promoter contained in the 3′ long terminal repeat of Rous sarcoma virus (Yamamoto et al., Cell, 22: 787-797, 1980); the herpes simplex thymidine kinase promoter (Wagner et al., Proc. Natl. Acad. Sci. USA, 78: 144-1445, 1981); the regulatory sequences of the metallothionein gene (Brinster et al., Nature, 296: 39-42, 1982); prokaryotic expression vectors such as the beta-lactamase promoter (Villa-Kamaroff, et al., Proc. Natl. Acad. Sci. USA, 75: 3727-3731, 1978); or the tac promoter (DeBoer, et al., Proc. Natl. Acad. Sci. USA, 80: 21-25, 1983). Also of interest are the following animal transcriptional-control regions, which exhibit tissue specificity and have been utilized in transgenic animals: the elastase I gene control region which is active in pancreatic acinar cells (Swift et al., Cell, 38: 639-646, 1984; Ornitz et al., Cold Spring Harbor Symp. Quant. Biol., 50:399-409, 1986; MacDonald, Hepatology, 7: 425-515, 1987); the insulin gene control region which is active in pancreatic beta cells (Hanahan, Nature, 315: 115-122, 1985); the immunoglobulin gene control region which is active in lymphoid cells (Grosschedl et al., Cell, 38: 647-658, 1984; Adames et al., Nature, 318: 533-538, 1985; Alexander et al., Mol. Cell. Biol., 7: 1436-1444, 1987); the mouse mammary tumor virus control region which is active in testicular, breast, lymphoid and mast cells (Leder et al., Cell, 45: 485-495, 1986); the albumin gene control region which is active in liver (Pinkert et al., Genes and Devel., 1: 268-276, 1987); the alphafetoprotein gene control region which is active in liver (Krumlauf et al., Mol. Cell. Biol., 5: 1639-1648, 1985; Hammer et al., Science, 235: 53-58, 1987); the alpha 1-antitrypsin gene control region which is active in the liver (Kelsey et al., Genes and Devel., 1: 161-171, 1987); the beta-globin gene control region which is active in myeloid cells (Mogram et al., Nature, 315: 338-340, 1985; Kollias et al., Cell, 46: 89-94, 1986); the myelin basic protein gene control region which is active in oligodendrocyte cells in the brain (Readhead et al., Cell, 48: 703-712, 1987); the myosin light chain-2 gene control region which is active in skeletal muscle (Sani, Nature, 314: 283-286, 1985); and the gonadotropic releasing hormone gene control region which is active in the hypothalamus (Mason et al., Science, 234: 1372-1378, 1986).

An enhancer sequence may be inserted into the vector to increase the transcription of a DNA encoding a Cdk11 polypeptide of the present invention by higher eukaryotes. Enhancers are cis-acting elements of DNA, usually about 10-300 bp in length, that act on the promoter to increase its transcription. Enhancers are relatively orientation and position independent. They have been found 5′ and 3′ to the transcription unit. Several enhancer sequences available from mammalian genes are known e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to Cdk11 nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art.

Preferred vectors for practicing this invention are those which are compatible with bacterial, insect, and mammalian host cells. Such vectors include, inter alia, pCR11, pCR3, and pcDNA3.1 (Invitrogen Company, Carlsbad, Calif.), pBSII (Stratagene Company, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO90/14363) and pFastBac1, pFastBacHT and pFastBacDual (Gibco/BRL, Grand Island, N.Y.).

Additional suitable vectors include, but are not limited to, cosmids, plasmids, or modified viruses, but it will be appreciated that the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™ TA Cloning® Kit, PCR2.1® plasmid derivatives, Invitrogen, Carlsbad, Calif.), and mammalian, yeast, or virus vectors such as a baculovirus expression system (pBacPAK plasmid derivatives, Clontech, Palo Alto, Calif.).

After the vector has been constructed and a nucleic acid molecule encoding a Cdk11 polypeptide has been inserted into the proper site of the vector, the completed vector may be inserted into a suitable host cell for amplification and/or polypeptide expression. The transformation of an expression vector for a Cdk11 polypeptide into a selected host cell may be accomplished by well-known methods such as transfection, infection, calcium chloride-mediated transformation, electroporation, microinjection, lipofection or the DEAE-dextran method or other known techniques. The method selected will in part be a function of the type of host cell to be used. These methods and other suitable methods are well-known to the skilled artisan and are set forth, for example, in Sambrook et al., supra.

Host cells may be prokaryotic host cells (such as E. coli) or eukaryotic host cells (such as a yeast, insect, or vertebrate cells). The host cell, when cultured under appropriate conditions, may synthesize a Cdk11 polypeptide which can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity, such as glycosylation or phosphorylation, and ease of folding into a biologically active molecule.

A number of suitable host cells are known in the art and many are available from the American Type Culture Collection (ATCC), 10801 University Boulevard, Manassas, Va. 20110-2209. Examples include, but are not limited to, mammalian cells, such as Chinese hamster ovary cells (CHO) (ATCC No. CCL61); CHO DHFR-cells (Urlaub et al., Proc. Natl. Acad. Sci. USA, 97: 4216-4220, 1980), human embryonic kidney (HEK) 293 or 293T cells (ATCC No. CRL1573); or 3T3 cells (ATCC No. CCL92). The selection of suitable mammalian host cells and methods for transformation, culture, amplification, screening, product production and purification are known in the art. Other suitable mammalian cell lines, are the monkey COS-1 (ATCC No. CRL1650) and COS-7 (ATCC No. CRL1651) cell lines, and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection-gene, or may contain a dominantly acting selection gene. Other suitable mammalian cell lines include, but are not limited to, mouse neuroblastoma N2A cells, HeLa, mouse L-929 cells, 3T3 lines derived from Swiss, Balb-c or NIH mice, BHK or HaK hamster cell lines, which are also available from the ATCC. Each of these cell lines is known by and available to those skilled in the art of protein expression.

Similarly useful as host cells suitable for the present invention are bacterial cells. For example, the various strains of E. coli (e.g., HB101, (ATCC No. 33694) DH5α, DH10, and MC1061 (ATCC No. 53338)) are well-known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for expression of the polypeptides of the present invention. Preferred yeast cells include, for example, Saccharomyces cerivisae and Pichia pastoris.

Additionally, where desired, insect cell systems may be utilized in the methods of the present invention. Such systems are described for example in Kitts et al., Biotechniques, 14: 810-817, 1993; Luckow, Curr. Opin. Biotechnol., 4: 564-572, 1993; and Luckow et al., J. Virol., 67: 4566-4579, 1993. Preferred insect cells are Sf-9 and Hi5 (Invitrogen, Carlsbad, Calif.). Preferably, the insect cells are infected with recombinant baculovirus particles which have the Cdk11 polynucleotide sequence incorporated into its genome. The baculovirus infection leads to efficient eukaryotic expression of recombinant Cdk11. Such baculovirus expression systems include the Bac-to-Bac® System (Life Technologies) and the BacPAK™ System (Clontech).

One may also use transgenic animals to express glycosylated Cdk11 polypeptides. For example, one may use a transgenic milk-producing animal (a cow or goat, for example) and obtain the present glycosylated polypeptide in the animal milk. One may also use plants to produce Cdk11 polypeptides.

Polypeptide Production

Host cells comprising a Cdk11 polypeptide expression vector may be cultured using standard media well known to the skilled artisan. The media will usually contain all nutrients necessary for the growth and survival of the cells. Suitable media for culturing E. coli cells include, for example, Luria Broth (LB) and/or Terrific Broth (TB). Suitable media for culturing eukaryotic cells include, Roswell Park Memorial Institute medium 1646 (RPMI 1640), Minimal Essential Medium (MEM), and/or Dulbecco's Modified Eagle Medium (DMEM), all of which may be supplemented with serum and/or growth factors as indicated for the particular cell line being cultured. A suitable medium for insect cultures is Grace's medium supplemented with yeastolate, lactalbumin hydrolysate, and/or fetal calf serum, as necessary.

Typically, an antibiotic or other compound useful for selective growth of transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

The amount of a Cdk 11 polypeptide produced by a host cell can be evaluated using standard methods known in the art. Such methods include, without limitation, Western blot analysis, SDS-polyacrylamide gel electrophoresis, chromatographic separation such as non-denaturing gel electrophoresis, High Performance Liquid Chromatography (HPLC), immunodetection such as immunoprecipitation, and/or activity assays such as DNA binding gel shift assays.

If a Cdk11 polypeptide has been designed to be secreted from the host cells, the majority of polypeptide may be found in the cell culture medium. If however, the Cdk11 polypeptide is not secreted from the host cells, it will be present in the cytoplasm and/or the nucleus (for eukaryotic host cells) or in the cytosol (for bacterial host cells).

For a Cdk11 polypeptide situated in the host cell cytoplasm and/or the nucleus (for eukaryotic host cells) or in the cytosol (for bacterial host cells), intracellular material (including inclusion bodies for gram-negative bacteria) can be extracted from the host cell using any standard technique known to the skilled artisan. For example, the host cells can be lysed to release the contents of the periplasm/cytoplasm by osmotic shock French press, homogenization, enzymatic disruption, exposure to deterdents or chaotropes, and/or sonication followed by centrifugation.

If a Cdk11 polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The Cdk11 polypeptide in its now soluble form can then be analyzed using gel electrophoresis, immunoprecipitation or the like. If it is desired to isolate the Cdk11 polypeptide, isolation may be accomplished using standard methods such as those described herein and in Marston et al., Meth. Enzymol., 182:264-275. 1990).

In some cases, a Cdk11 polypeptide may not be biologically active upon isolation. Various methods for “refolding” or converting the polypeptide to its tertiary structure and generating disulfide linkages can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol (DTT)/dithiane DTT, and 2-mercaptoethanol (βME)/dithio-β(ME). A cosolvent may be used to increase the efficiency of the refolding, and the more common reagents used for this purpose include glycerol, polyethylene glycol of various molecular weights, arginine and the like.

If inclusion bodies are not formed to a significant degree upon expression of a Cdk11 polypeptide, then the polypeptide will be found primarily in the supernatant after centrifugation of the cell homogenate. The polypeptide may be further isolated from the supernatant using methods such as those described herein or otherwise known in the art.

The purification of a Cdk11 polypeptide from solution can be accomplished using a variety of techniques. If the polypeptide has been synthesized such that it contains a tag such as Hexahistidine (Cdk11 polypeptide/hexaHis) or other small peptide such as FLAG (Eastman Kodak Co., New Haven, Conn.) or myc (Invitrogen, Carlsbad, Calif.) at either its carboxyl or amino terminus, it may be purified in a one-step process by passing the solution through an affinity column where the column matrix has a high affinity for the tag.

For example, polyhistidine binds with great affinity and specificity to nickel; thus an affinity column of nickel (such as the Qiagen® nickel columns) can be used for purification of Cdk11 polypeptide/polyHis. See for example, Ausubel et al., eds., Current Protocols in Molecular Biology, Section 10.11.8, John Wiley & Sons, New York (1993).

Additionally, the Cdk11 polypeptide may be purified through use of a monoclonal antibody which is capable of specifically recognizing and binding to the Cdk11 polypeptide.

Suitable procedures for purification thus include, without limitation, affinity chromatography, immunoaffinity chromatography, ion exchange chromatography, molecular sieve chromatography, High Performance Liquid Chromatography (HPLC), electrophoresis (including native gel electrophoresis) followed by gel elution, and preparative isoelectric focusing (“Isoprime” machine/technique, Hoefer Scientific, San Francisco, Calif.). In some cases, two or more of these purification techniques may be combined to achieve increased purity.

Cdk11 polypeptides may also be prepared by chemical synthesis methods (such as solid phase peptide synthesis) using techniques known in the art, such as those set forth by Merrifield et al., J. Am. Chem. Soc., 85:2149, 1963, Houghten et al., Proc. Natl. Acad. Sci. USA, 82:5132, 1985, and Stewart and Young, Solid Phase Peptide Synthesis, Pierce Chemical Co., Rockford, Ill., 1984. Such polypeptides may be synthesized with or without a methionine on the amino terminus. Chemically synthesized Cdk11 polypeptides may be oxidized using methods set forth in these references to form disulfide bridges. Chemically synthesized Cdk11 polypeptides are expected to have comparable biological activity to the corresponding Cdk11 polypeptides produced recombinantly or purified from natural sources, and thus may be used interchangeably with a recombinant or natural Cdk11 polypeptide.

Another means of obtaining a Cdk 11 polypeptide is via purification from biological samples such as source tissues and/or fluids in which the Cdk11 polypeptide is naturally found. Such purification can be conducted using methods for protein purification as described herein or as otherwise known in the art. The presence of the Cdk11 polypeptide during purification may be monitored using, for example, using an antibody prepared against recombinantly produced Cdk11 polypeptide or peptide fragments thereof.

A number of additional methods for producing nucleic acids and polypeptides are known in the art, and the methods can be used to produce polypeptides having specificity for Cdk11. See for example, Roberts et al., Proc. Nail. Acad. Sci. U.S.A., 94:12297-12303, 1997, which describes the production of fusion proteins between an mRNA and its encoded peptide. See also Roberts, R., Curr. Opin. Chem. Biol., 3:268-273, 1999. Additionally, U.S. Pat. No. 5,824,469 describes methods of obtaining oligonucleotides capable of carrying out a specific biological function. The procedure involves generating a heterogeneous pool of oligonucleotides, each having a 5′ randomized sequence, a central preselected sequence, and a 3′ randomized sequence. The resulting heterogeneous pool is introduced into a population of cells that do not exhibit the desired biological function. Subpopulations of the cells are then screened for those which exhibit a predetermined biological function. From that subpopulation, oligonucleotides capable of carrying out the desired biological function are isolated.

U.S. Pat. Nos. 5,763,192; 5,814,476; 5,723,323; and 5,817,483 describe processes for producing peptides or polypeptides. This is done by producing stochastic genes or fragments thereof, and then introducing these genes into host cells which produce one or more proteins encoded by the stochastic genes. The host cells are then screened to identify those clones producing peptides or polypeptides having the desired activity.

Another method for producing peptides or polypeptides is described in PCT/US98/20094 (WO99/15650) filed by Athersys, Inc. Known as “Random Activation of Gene Expression for Gene Discovery” (RAGE-GD), the process involves the activation of endogenous gene expression or over-expression of a gene by in situ recombination methods. For example, expression of an endogenous gene is activated or increased by integrating a regulatory sequence into the target cell which is capable of activating expression of the gene by non-homologous or illegitimate recombination. The target DNA is first subjected to radiation, and a genetic promoter inserted. The promoter randomly locates a break at the front 5′ end of a gene, initiating transcription of the gene. This results in expression of the desired peptide or polypeptide.

It will be appreciated that these methods can also be used to create comprehensive Cdk11 protein expression libraries, which can subsequently be used for high throughput phenotypic screening in a variety of assays, such as biochemical assays, cellular assays, and whole organism assays (e.g., plant, mouse, etc.).

Chemical Derivatives

Chemically modified derivatives of the Cdk11 polypeptides may be prepared by one skilled in the art, given the disclosures set forth herein below. Cdk11 polypeptide derivatives are modified in a manner that is different, either in the type or location of the molecules naturally attached to the polypeptide. Derivatives may include molecules formed by the deletion of one or more naturally-attached chemical groups. The polypeptide comprising the amino acid sequence of SEQ ID NO: 2, or a Cdk11 polypeptide variant, may be modified by the covalent attachment of one or more polymers. For example, the polymer selected is typically water soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water soluble polymer, some molecules will weigh more, some less, than the stated molecular weight). The average molecular weight of each polymer is preferably between about 5 kDa and about 50 kDa, more preferably between about 12 kDa and about 40 kDa and most preferably between about 20 kDa to about 35 kDa. Suitable water soluble polymers or mixtures thereof include, but are not limited to, N-linked or O-linked carbohydrates; sugars; phosphates; polyethylene-glycol (PEG) (including the forms of PEG that have been used to derivatize proteins, including mono-(C1-C10) alkoxy- or aryloxy-polyethylene glycol), monomethoxy-polyethylene glycol; dextran (such as low molecular weight dextran of, for example about 6 kD, cellulose, or other carbohydrate-based polymers, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional crosslinking molecules which may be used to prepare covalently attached multimers of the polypeptide comprising the amino acid sequence of SEQ ID NO: 2 or a Cdk11 polypeptide variant.

In general, chemical derivatization may be performed under any suitable condition used to react a protein with an activated polymer molecule. Methods for preparing chemical derivatives of polypeptides will generally comprise the steps of (a) reacting the polypeptide with the activated polymer molecule (such as a reactive ester or aldehyde derivative of the polymer molecule) under conditions whereby the polypeptide comprising the amino acid sequence of SEQ ID NO: 2, or a Cdk11 polypeptide variant becomes attached to one or more polymer molecules, and (b) obtaining the reaction product(s). The optimal reaction conditions will be determined based on known parameters and the desired result. For example, the larger the ratio of polymer molecules:protein, the greater the percentage of attached polymer molecule. In one embodiment, the Cdk11 polypeptide derivative may have a single polymer molecule moiety at the amino terminus. (See, for example, U.S. Pat. No. 5,234,784).

The pegylation of the polypeptide may be specifically carried out by any of the pegylation reactions known in the art, as described for example in the following 115 references: Francis et al., Focus on Growth Factors, 3:4-10 (1992); EP 0154316; EP 0401384 and U.S. Pat. No. 4,179,337. For example, pegylation may be carried out via an acylation reaction or an alkylation reaction with a reactive polyethylene glycol molecule (or an analogous reactive water-soluble polymer) as described herein. For the acylation reactions, the polymer(s) selected should have a single reactive ester group. For reductive alkylation, the polymer(s) selected should have a single reactive aldehyde group. A reactive aldehyde is, for example, polyethyleneglycol propionaldehyde, which is water stable, or mono C1-C10 alkoxy or aryloxy derivatives thereof (see U.S. Pat. No. 5,252,714).

In another embodiment, Cdk11 polypeptides may be chemically coupled to biotin, and the biotin/Cdk11 polypeptide molecules which are conjugated are then allowed to bind to avidin, resulting in tetravalent avidin/biotin/Cdk11 polypeptide molecules. Cdk11 polypeptides may also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions which may be alleviated or modulated by the administration of the present Cdk11 polypeptide derivatives include those described herein for Cdk11 polypeptides. However, the Cdk11 polypeptide derivatives disclosed herein may have additional activities, enhanced or reduced biological activity, or other characteristics, such as increased or decreased half-life, as compared to the non-derivatized molecules.

Genetically Engineered Non-Human Animals

Additionally included within the scope of the present invention are non-human animals such as mice, rats, rabbits or other rodents, goats, or sheep, or other farm animals, in which the gene (or genes) encoding the native Cdk11 polypeptide has (have) been disrupted (“knocked out”) such that the level of expression of this gene or genes is (are) significantly decreased or completely abolished. Such animals may be prepared using techniques and methods such as those described in U.S. Pat. No. 5,557,032.

The present invention further includes non-human animals such as mice, rats, rabbits, or other rodents, rabbits, goats, sheep, or other farm animals, in which either the native form of the cdk11 gene(s) for that animal or a heterologous Cdk11 gene(s) is fare) over-expressed by the animal, thereby creating a “transgenic” animal. Such transgenic animals may be prepared using well known methods such as those described in U.S. Pat. No. 5,489,743 and PCT Application No. WO 94/28122.

The present invention further includes non-human animals in which the promoter for one or more of the Cdk11 polypeptides of the present invention is either activated or inactivated (e.g., by using homologous recombination methods) to alter the level of expression of one or more of the native Cdk11 polypeptides.

These non-human animals may be used for drug candidate screening in such screening, the impact of a drug candidate on the animal is measured; for example, drug candidates may decrease or increase the expression of the Cdk11 gene. In certain embodiments, the amount of Cdk11 polypeptide, that is produced is measured after the exposure of the animal to the drug candidate. Additionally, in certain embodiments, one may detect the actual impact of the drug candidate on the animal. For example, the overexpression of a particular gene may result in, or be associated with, a disease or pathological condition. In such cases, one may test a drug candidate's ability to decrease expression of the gene or its ability to prevent, inhibit or eliminate a pathological condition. In other examples, the production of a particular metabolic product such as a fragment of a polypeptide, may result in, or be associated with, a disease or pathological condition. In such cases, one may test a drug candidate's ability to decrease the production of such a metabolic product or its ability to prevent, inhibit or eliminate a pathological condition.

Microarray

It will be appreciated that DNA microarray technology can be utilized in accordance with the present invention. DNA microarrays are miniature, high density arrays of nucleic acids positioned on a solid support, such as glass. Each cell or element within the array has numerous copies of a single species of DNA which acts as a target for hybridization for its cognate mRNA. In expression profiling using DNA microarray technology, mRNA is first extracted from a cell or tissue sample and then converted enzymatically to fluorescently labeled cDNA. This material is hybridized to the microarray and unbound cDNA is removed by washing. The expression of discrete genes represented on the array is then visualized by quantitating the amount of labeled cDNA which is specifically bound to each target DNA. In this way, the expression of thousands of genes can be quantitated in a high throughput, parallel manner from a single sample of biological material.

This high throughput expression profiling has a broad range of applications with respect to the Cdk11 molecules of the invention, including, but not limited to: the identification and validation of Cdk11 disease-related genes as targets for therapeutics; molecular toxicology of Cdk11 molecules and inhibitors thereof; stratification of populations and generation of surrogate markers for clinical trials; and the enhancement of an Cdk11-related small molecule drug discovery by aiding in the identification of selective compounds in high throughput screens (HTS).

Selective Binding Agents

As used herein, the term “selective binding agent” refers to a molecule which has specificity for one or more Cdk11 polypeptides. Suitable selective binding agents include, but are not limited to, antibodies and derivatives thereof, polypeptides, and small molecules. Suitable selective binding agents may be prepared using methods known in the art. An exemplary Cdk11 polypeptide selective binding agent of the present invention is capable of binding a certain portion of the Cdk11 polypeptide, thereby inhibiting the activity or function of Cdk11 polypeptide.

Selective binding agents such as antibodies and antibody fragments that bind Cdk11 polypeptides are within the scope of the present invention. The antibodies may be polyclonal including monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody which bind to an epitope on the Cdk11 polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward a cdk11 polypeptide generally are produced in animals (e.g., rabbits or mite) by means of multiple subcutaneous, intramuscular or intraperitoneal injections of Cdk11 polypeptide and an adjuvant. It may be useful to conjugate a Cdk11 polypeptide to a carrier protein that is immunogenic in the species to be immunized, such as keyhole limpet hemocyanin, serum, albumin, bovine thyroglobulin, or soybean trypsin inhibitor. Also, aggregating agents such as alum are used to enhance the immune response. After immunization, the animals are bled and the serum is assayed for anti-Cdk11 polypeptide antibody titer.

Monoclonal antibodies directed toward Cdk11 polypeptide are produced using any method which provides for the production of antibody molecules by continuous cell lines in culture. Examples of suitable methods for preparing monoclonal antibodies include the hybridoma methods of Kohler et al. (Nature, 256: 495-497, 1975) and the human B-cell hybridoma method, Kozbor (J. Immunol., 133: 3001, 1984; Brodeur et al., Monoclonal Antibody Production Techniques and Applications, pp. 51-63, Marcel Dekker, Inc., New York, 1987). Also provided by the invention are hybridoma cell lines which produce monoclonal antibodies reactive with Cdk11 polypeptides.

Monoclonal antibodies of the invention may be modified for use as therapeutics. One embodiment is a “chimeric” antibody in which a portion of the heavy and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See, U.S. Pat. No. 4,816,567 and Morrison et al., Proc. Natl. Acad. Sci. U.S.A., 81: 6851-6855 (1985).

In another embodiment, a monoclonal antibody of the invention is a “humanized” antibody. Methods for humanizing non-human antibodies are well known in the art. (see U.S. Pat. Nos. 5,585,089, and 5,693,762). Generally, a humanized antibody has one or more amino acid residues introduced into it from a source which is non-human. Humanization can be performed, for example, using methods described in the art (Jones et al., Nature 321: 522-525, 1986; Riechmann et al., Nature, 332:323-327, 1988; Verhoeyen et al., Science 239:1534-1536, 1988), by substituting at least a portion of a rodent complementarity-determining region (CDR) for the corresponding region of a human antibody.

Also encompassed by the invention are human antibodies which bind Cdk11 polypeptide, fragments, variants and/or derivatives. Using transgenic animals (e.g., mice) that are capable of producing a repertoire of human antibodies in the absence of endogenous immunoglobulin production such antibodies are produced by immunization with a Cdk11 antigen (i.e., having at least 6 contiguous amino acids), optionally conjugated to a carrier. See, for example, Jakobovits et al., Proc. Natl. Acad. Sci. USA., 90: 2551-2555, 1993; Jakobovits et al., Nature 362: 255-258, 1993; Bruggermann et al., Year in Immunol., 7: 33, 1993. In one method, such transgenic animals are produced by incapacitating the endogenous loci encoding the heavy and light immunoglobulin chains therein, and inserting loci encoding human heavy and light chain proteins into the genome thereof. Partially modified animals, that is those having less than the full complement of modifications, are then cross-bred to obtain an animal having all of the desired immune system modifications. When administered an immunogen, these transgenic animals produce antibodies with human variable regions, including human (rather than e.g., murine) amino acid sequences, including variable regions which are immunospecific for these antigens. See PCT Application Nos. PCT/US96/05928 and PCT/US93/06926. Additional methods are described in U.S. Pat. No. 5,545,807, PCT Application Nos. PCT/US91/245, PCT/GB89/01207, and in EP 546073B1 and EP 546073A1. Human antibodies may also be produced by the expression of recombinant DNA in host cells or by expression in hybridoma cells as described herein.

In an alternative embodiment, human antibodies can be produced from phage-display libraries (Hoogenboom et al., J. Mol. Biol. 227: 381 1991; Marks et al., J. Mol. Biol. 222: 581, 1991). These processes mimic immune selection through the display of antibody repertoires on the surface of filamentous bacteriophage, and subsequent identification of phage by their binding to an antigen of choice. One such technique is described in PCT Application No. PCT/US98/17364, filed in the name of Adams et al., which describes the isolation of high affinity and functionally agonistic antibodies for MPL- and msk-receptors using such an approach.

Chimeric, CDR grafted, and humanized antibodies are typically produced by recombinant methods. Nucleic acids encoding the antibodies are introduced into host cells and expressed using materials and procedures described herein or known in the art. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

The anti-Cdk11 antibodies of the invention may be employed in any known assay method, such as competitive binding assays, direct and indirect sandwich assays, and immunoprecipitation assays, (Sola, Monoclonal Antibodies: A Manual of Techniques, pp. 147-158 (CRC Press, Inc., 1987)) for the detection and quantitation of Cdk11 polypeptides. The antibodies will bind Cdk11 polypeptides with an affinity which is appropriate for the assay method being employed.

For diagnostic applications, in certain embodiments, anti-Cdk11 antibodies typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase, or horseradish peroxidase (Bayer et al., Meth. Enzymol., 184: 138-163, 1990).

Competitive binding assays rely on the ability of a labeled standard (e.g., a Cdk11 polypeptide, or an immunologically reactive portion thereof) to compete with the test sample analyte (a Cdk11 polypeptide) for binding with a limited amount of anti-Cdk11 antibody. The amount of a Cdk11 polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte which remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The selective binding agents, including anti-Cdk11 antibodies, are also useful for in vivo imaging. An antibody labeled with a detectable moiety may be administered to an animal, preferably into the bloodstream, and the presence and location of the labeled antibody in the host is assayed. The antibody may be labeled with any moiety that is detectable in an animal, whether by nuclear magnetic resonance, radiology, or other detection means known in the art.

Selective binding agents of the invention, including anti-Cdk11 antibodies, may be used as therapeutics. These therapeutic agents are generally agonists or antagonists, in that they either enhance or reduce, respectively, at least one of the biological activities of a Cdk11 polypeptide. In one embodiment, antagonist antibodies of the invention are antibodies or binding fragments thereof which are capable of specifically binding to a Cdk11 polypeptide and which are capable of inhibiting or eliminating the functional activity of a Cdk11 polypeptide in vivo or in vitro. In preferred embodiments, the selective binding agent, e.g., an antagonist antibody will inhibit the functional activity of a Cdk11 polypeptide by at least about 50%, and preferably by at least about 80%. In another embodiment, the selective bingeing agent may be an antibody that is capable of interacting with a Cdk11 binding partner (a ligand, co-factor, or receptor) thereby inhibiting or eliminating Cdk11 activity in vitro or in vivo. Selective binding agents, including agonist and antagonist anti-Cdk11 antibodies are identified by screening assays which are well known in the art.

The invention also relates to a kit comprising Cdk11 selective binding agents (such as antibodies) and other reagents useful for detecting Cdk11 levels in biological samples. Such reagents may include a secondary activity, a detectable label, blocking serum, positive and negative control samples, and detection reagents.

Cdk11 polypeptides can be used to clone Cdk11 binding partners using an “expression cloning” strategy. Radiolabeled (¹²⁵I) Cdk11 polypeptide or “affinity/activity-tagged” Cdk11 polypeptide (such as an Fc fusion or an alkaline phosphatase fusion) can be used in binding assays to identify a cell type or a cell line or tissue that expresses Cdk11 binding partners. RNA isolated from such cells or tissues can then be converted to cDNA, cloned into a mammalian expression vector, and transfected into mammalian cells (for example, COS, or 293) to create an expression library. Radiolabeled or tagged Cdk11 polypeptide can then be used as an affinity reagent to identify and isolate the subset of cells in this library expressing Cdk11 binding partners. DNA is then isolated from these cells and transfected into mammalian cells to create a secondary expression library in which the fraction of cells expressing Cdk11 binding partners would be many-fold higher than in the original library. This enrichment process can be repeated iteratively until a single recombinant clone containing an Cdk11 binding partner is isolated. Isolation of Cdk11 binding partners is useful for identifying or developing novel agonists and antagonists of the Cdk11 signaling pathway. Such agonists and antagonists include Cdk11 binding partners, cyclins, Cdk inhibitors, Cdk cofactors, anti-Cdk11 binding partner antibodies, small molecules or antisense oligonucleotides.

Assaying for other Modulators of Cdk11 Polypeptide Activity

In some situations, it may be desirable to identify molecules that are modulators, i.e., agonists or antagonists, of the activity of Cdk11 polypeptide. Natural or synthetic molecules that modulate Cdk11 polypeptide may be identified using one or more screening assays, such as those described herein. Such molecules may be administered either in an ex vivo manner, or in an in vivo manner by injection, or by oral delivery, implantation device, or the like. “Test molecule(s)” refers to the molecule(s) that is/are under evaluation for the ability to modulate (i.e., increase or decrease) an activity of a Cdk11 polypeptide. Most commonly, a test molecule will interact directly with a Cdk11 polypeptide. However, it is also contemplated that a test molecule may also modulate Cdk11 polypeptide activity indirectly, such as by affecting Cdk11 gene expression, or by binding to a Cdk11 binding partner (e.g., receptor, co-factor, or ligand). In one embodiment, a test molecule will bind to a Cdk11 polypeptide with an affinity constant of at least about 10⁻⁶ M, preferably about 10⁻⁸ M, more preferably about 10⁻⁹ M, and even more preferably about 10⁻¹⁰ M.

Methods for identifying compounds which interact with Cdk11 polypeptides are encompassed by the present invention. In certain embodiments, an Cdk11 polypeptide is incubated with a test molecule under conditions which permit the interaction of the test molecule with a Cdk11 polypeptide, and the extent of the 2-0 interaction can be measured. The test molecule(s) can be screened in a substantially purified form or in a crude mixture.

In certain embodiments, a Cdk11 polypeptide agonist or antagonist may be a protein, peptide, carbohydrate, lipid, or small molecular weight molecule which interacts with Cdk11 polypeptide to regulate its activity. Molecules which regulate Cdk11 polypeptide expression include nucleic acids which are complementary to nucleic acid encoding a Cdk11 polypeptide, or are complementary to nucleic acids sequences which direct or control the expression of Cdk11 polypeptide, and which act as anti-sense regulators of expression.

Once a set of test molecules has been identified as interacting with a Cdk11 polypeptide, the molecules may be further evaluated for their ability to increase or decrease Cdk11 polypeptide activity. The measurement of the interaction of test molecules with Cdk11 polypeptides may be carried out in several formats, including cell-based binding assays, membrane binding assays, solution-phase assays and immunoassays. In general, test molecules are incubated with a Cdk11 polypeptide for a specified period of time, and Cdk11 polypeptide activity is determined by one or more assays for measuring biological activity.

The interaction of test molecules with Cdk11 polypeptides may also be assayed directly using polyclonal or monoclonal antibodies in an immunoassay. Alternatively, modified forms of Cdk11 polypeptides containing epitope tags as described herein may be used in immunoassays.

Cdk11 polypeptides displaying biological activity through an interaction with a binding partner (e.g., a cyclin or a co-factor), are assessed by a variety of in vitro assays that may be used to measure the binding of a Cdk11 polypeptide to the corresponding binding partner (such as a selective binding agent, cyclin, or co-factor). These assays are used to screen test molecules for their ability to increase or decrease the rate and/or the extent of binding of a Cdk11 polypeptide to its binding partner. In one assay, a Cdk11 polypeptide is immobilized in the wells of a microtiter plate. Radiolabeled Cdk 11 binding partner (for example, iodinated Cdk 11 binding partner) and the test molecule(s) are added either one at a time (in either order) or simultaneously to the wells. After incubation, the wells is washed and counted using a scintillation counter, for radioactivity to determine the extent to which the binding partner bound to Cdk11 polypeptide. Typically, the molecules will be tested over a range of concentrations, and a series of control wells lacking one or more elements of the test assays are used for accuracy in the evaluation of the results. An alternative to this method involves reversing the “positions” of the proteins, i.e., immobilizing Cdk11 binding partner to the microtiter plate wells, incubating with the test molecule and radiolabeled Cdk11 polypeptide, and determining the extent of Cdk11 polypeptide binding. See, for example, chapter 18, Current Protocols in Molecular Biology, Ausubel et al., eds., John Wiley & Sons, New York, N.Y. (1995).

As an alternative to radiolabelling, a Cdk11 polypeptide or its binding partner may be conjugated to biotin and the presence of biotinylated protein can then be detected using streptavidin linked to an enzyme, such as horseradish peroxidase (HRP) or alkaline phosphatase (AP), that is detected colorometrically, or by fluorescent tagging of streptavidin. An antibody directed to a Cdk11 polypeptide or to a Cdk11 binding partner and conjugated to biotin may also be used and is detected after incubation with enzyme-linked streptavidin linked to AP or HRP.

A Cdk11 polypeptide or a Cdk11 binding partner can also be immobilized by attachment to agarose beads, acrylic beads or other types of such inert solid phase substrates. The substrate-protein complex is placed in a solution containing the complementary protein and the test compound. After incubation, the beads can be precipitated by centrifugation, and the amount of binding between a Cdk11 polypeptide and its binding partner is assessed using the methods described herein. Alternatively, the substrate-protein complex is immobilized in a column, and the test molecule and complementary protein are passed through the column. The formation of a complex between a Cdk11 polypeptide and its binding partner is assessed using any of the techniques set forth herein, i.e., radiolabelling, antibody binding or the like.

Another in vitro assay that is useful for identifying a test molecule that increases or decreases the formation of a complex between a Cdk11 polypeptide and a Cdk11 binding partner is a surface plasmon resonance detector system such as the BIAcore assay system (Pharmacia, Piscataway, N.J.). The BIAcore system may be carried out using the manufacturer's protocol. This assay essentially involves the covalent binding of either Cdk11 polypeptide or a Cdk11 binding partner to a dextran-coated sensor chip which is located in a detector. The test compound and the other complementary protein can then be injected, either simultaneously or sequentially, into the chamber containing the sensor chip. The amount of complementary protein that binds can be assessed based on the change in molecular mass which is physically associated with the dextran-coated side of the sensor chip. The change in molecular mass can be measured by the detector system.

In some cases, it may be desirable to evaluate two or more test compounds together for their ability to increase or decrease the formation of a complex between a Cdk11 polypeptide and a Cdk11 binding partner. In these cases, the assays set forth herein can be readily modified by adding such additional test compound(s) either simultaneous with, or subsequent to, the first test compound. The remainder of the steps in the assay are set forth herein.

In vitro assays such as those described herein may be used advantageously to screen large numbers of compounds for effects on complex formation by Cdk11 polypeptide and Cdk11 binding partner. The assays may be automated to screen compounds generated in phage display, synthetic peptide, and chemical synthesis libraries.

Compounds which increase or decrease the formation of a complex between a Cdk11 polypeptide and a Cdk11 binding partner may also be screened in cell culture using cells and cell lines expressing either Cdk11 polypeptide or Cdk11 binding partner. Cells and cell lines may be obtained from any mammal, but preferably will be from human or other primate, canine, or rodent sources. The binding of a Cdk11 polypeptide to cells expressing Cdk11 binding partner at the surface is evaluated in the presence or absence of test molecules, and the extent of binding may be determined by, for example, flow cytometry using a biotinylated antibody to a Cdk11 binding partner. Cell culture assays can be used advantageously to further evaluate compounds that score positive in protein binding assays described herein.

Cell cultures can also be used to screen the impact of a drug candidate. For example, drug candidates may decrease or increase the expression of the Cdk11 gene. In certain embodiments, the amount of Cdk11 polypeptide that is produced may be measured after exposure of the cell culture to the drug candidate. In certain embodiments, one may detect the actual impact of the drug candidate on the cell culture. For example, the overexpression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug candidate's ability to decrease the production of such a metabolic product in a cell culture.

A yeast two-hybrid system (Chien et al., Proc. Natl. Acad. Sci. USA, 88:9578-9583, 1991) can be used to identify novel polypeptides that bind to, or interact with, Cdk11 polypeptides. As an example, a yeast-two hybrid bait construct can be generated in a vector (such as the pAS2-1 from Clontech) which encodes a yeast GAL4-DNA binding domain fused to the Cdk11 polynucleotide. This bait construct may be used to screen human cDNA libraries wherein the cDNA library sequences are fused to GALA activation domains. Positive interactions will result in the activation of a reporter gene such as β-Gal. Positive clones emerging from the screening may be characterized further to identify interacting proteins.

Internalizing Proteins

The TAT protein sequence (from HIV) can be used to internalize proteins into a cell by targeting the lipid bi-layer component of the cell membrane. See e.g., Falwell et al., Proc. Nail. Acad. Sci., 91: 664-668, 1994. For example, an 11 amino acid sequence (YGRKKRRQRRR; SEQ ID NO: 15) of the HIV TAT protein (termed the “protein transduction domain”, or TAT PDT) has been shown to mediate delivery of large bioactive proteins such as β-galactosidase and p27^{Kip} across the cytoplasmic membrane and the nuclear membrane of a cell. See Schwarze et al., Science, 285: 1569-1572, 1999; and Nagahara et al., Nature Medicine, 4: 1449-1452, 1998. Schwarze et al. (Science, 285: 1569-72, 1999) demonstrated that cultured cells acquired β-gal activity when exposed to a fusion of the TAT PDT and β-galactosidase. Injection of mice with the TAT-β-gal fusion proteins resulted in β-gal expression in a number of tissues, including liver, kidney, lung, heart, and brain tissue.

It will thus be appreciated that the TAT protein sequence may be used to internalize a desired protein or polypeptide into a cell. In the context of the present invention, the TAT protein sequence can be fused to another molecule such as a Cdk11 antagonist (i.e.: anti-Cdk11 selective binding agent or small molecule) and administered intracellularly to inhibit the activity of the Cdk11 molecule. Where desired, the Cdk11 protein itself, or a peptide fragment or modified form of Cdk11, may be fused to such a protein transducer for administrating to cells using the procedures, described above.

Therapeutic Use

In one aspect, the present invention provides reagents and methods useful for treating diseases and conditions characterized by aberrant level of Cdk11 activity in a cell. A non-exclusive list of acute and chronic diseases which can be treated, diagnosed ameliorated, or prevented with the polypeptides, nucleic acids, antibodies, and/or fragments thereof of the invention include hyperproliferative pathological condition such as immune disorders, angiogenesis, vasculogenesis, wound healing, diabetes mellitus including diabetes type I and type II, psoriasis, liver diseases such as hepatitis and cirrhosis, osteoporosis, inflammatory conditions such as osteoarthritis and rheumatoid arthritis, pregnancy and cancer. More specifically, the types of cancers that can be treated, diagnosed, ameliorated or prevented with Cdk11 polypeptides and/or nucleic acids include, but are not limited to, colorectal adenocarcinoma, lung adenocarcinoma, lung squamous cell carcinoma, prostate adenocarcinoma, ovarian carcinoma, uterine carcinoma, breast adenocarcinoma, melanoma, leukemia, lymphoma including Hodgkin's Disease and non-Hodgkin's lymphoma, and brain tumors such as glioma and neuroblastoma. For the treatment of cancers, Cdk11 antagonists could be used in combination with chemotherapy for a more effective cancer treatment.

Cdk11 antagonists may also be used a chemoprotective agents which protect normal cells from damage during radiation and chemotherapy. For example, the epithelial cells of the gut and bone marrow cells could be protected by Cdk11 antagonist which could reduce side effects in patients receiving radiation and chemotherapy. In addition, a cream comprising Cdk 11 antagonists could be used to prevent chemotherapy induced hair loss.

Other diseases or disorders caused or mediated by undesirable levels of Cdk11 polypeptide are encompassed within the therapeutic and diagnostic utilities that are part of the invention. By way of illustration, such undesirable levels include excessively elevated levels and sub-normal levels.

Cdk11 Compositions and Administration

Therapeutic compositions are within the scope of the present invention. Such Cdk11 pharmaceutical compositions that may comprise a therapeutically effective amount of a Cdk11 polypeptide or a Cdk11 nucleic acid molecule in admixture with a pharmaceutically or physiologically acceptable formulation agent selected for suitability with the mode of administration to a human or non-human animal such as a mammal. Pharmaceutical compositions may comprise a therapeutically effective amount of one or more Cdk11 selective binding agents in admixture with a pharmaceutically or physiologically acceptable formulation agent selected for suitability with the mode of administration.

Acceptable formulation materials preferably are nontoxic to recipients at the dosages and concentrations employed.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides (preferably sodium or potassium chloride, mannitol, or sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the Cdk11 molecule.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, Cdk11 polypeptide compositions may be prepared for storage by mixing the selected composition having the desired degree of purity with optional formulation agents (Remington's Pharmaceutical Sciences, supra.) in the form of a lyophilized cake or an aqueous solution. Further, the Cdk11 polypeptide product may be formulated as a lyophilizate using appropriate excipients such as sucrose.

The Cdk11 pharmaceutical compositions can be selected for parenteral delivery. Alternatively, the compositions may be selected for inhalation or for delivery through the digestive tract, such as orally, or through other delivery routes known in the art. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this invention may be in the form of a pyrogen-free, parenterally acceptable aqueous solution comprising the desired Cdk11 molecule in a pharmaceutically acceptable vehicle. A particularly suitable vehicle for parenteral injection is sterile distilled water in which a Cdk11 molecule is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, a Cdk11 molecule may be formulated as a dry powder for inhalation. Cdk11 polypeptide or Cdk11 nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, Cdk11 molecules which are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the Cdk11 molecule. Diluents, flavorings, low melting point waxes, vegetable oils, lubricants, suspending agents, tablet disintegrating agents, and binders may also be employed.

Another pharmaceutical composition may involve an effective quantity of Cdk11 molecules in a mixture with non-toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional Cdk11 pharmaceutical compositions will be evident to those skilled in the art, including formulations involving Cdk11 polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 which describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556, 1983), poly (2-hydroxyethyl-methacrylate) (Langer et al., J. Biomed. Mater. Res., 15:167-277, 1981) and Langer et al., Chem. Tech., 12:98-105, 1982), ethylene vinyl acetate (Langer et al., supra) or poly-D (−)-3-hydroxybutyric acid (EP 133,988). Sustained-release compositions also include liposomes, which can be prepared by any of several methods known in the art. See e.g., Eppstein et al., Proc. Natl. Acad. Sci. USA, 82:3688-3692, 1985; EP 36,676; EP 88,046; EP 143,949.

The Cdk 11 pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a Cdk11 pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the Cdk11 molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 100 mg/kg.

The frequency of dosing will depend upon the pharmacokinetic parameters of the Cdk11 molecule in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data, which is routinely obtained.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to onto which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use Cdk11 pharmaceutical compositions in an ex vivo manner. In such instances, cells, tissues, or organs that have been removed from the patient are exposed to Cdk11 pharmaceutical compositions after which the cells, tissues and/or organs are subsequently implanted back into the patient.

In other cases, a Cdk11 polypeptide can be delivered by implanting certain cells that have been genetically engineered, using methods such as those described herein, to express and secrete the polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent Cdk11 gene, or an under expressed gene, and thereby produce a cell which expresses therapeutically efficacious amounts of Cdk11 polypeptides.

Homologous recombination is a technique originally developed for targeting genes to induce or correct mutations in transcriptionally active genes (Kucherlapati et al., Prog. in Nucl. Acid Res. & Mol. Biol., 36:301, 1989). The basic technique was developed as a method for introducing specific mutations into specific regions of the mammalian genome (Thomas et al., Cell, 44:419-428, 1986; Thomas and Capecchi, Cell, 51:503-512, 1987; Doetschman et al., Proc. Natl. Acad. Sci., 85:8583-8587, 1988) or to correct specific mutations within defective genes (Doetschman et al., Nature, 330:576-578, 1987). Exemplary homologous recombination techniques are described in U.S. Pat. No. 5,272,071 (EP 9193051, EP Publication No. 505500; PCT/US90/07642, International Publication No. WO 91/09955).

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA which may interact with or control the expression of a Cdk11 polypeptide, e.g., flanking sequences. For example, a promoter/enhancer element, a suppressor, or an exogenous transcription modulatory element is inserted in the genome of the intended host cell in proximity and orientation sufficient to influence the transcription of DNA encoding the desired Cdk11 polypeptide. The control element controls a portion of the DNA present in the host cell genome. Thus, the expression of the desired Cdk11 polypeptide may be achieved not by transfection of DNA that encodes the Cdk11 gene itself, but rather by the use of targeting DNA (containing regions of homology with the endogenous gene of interest) coupled with DNA regulatory segments that provide the endogenous gene sequence with recognizable signals for transcription of a Cdk11 polypeptide.

In an exemplary method, the expression of a desired targeted gene in a cell (i.e., a desired endogenous cellular gene) is altered via recombination into the cellular genome at a preselected site, by the introduction of DNA which includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which recombination can be used to increase, or cause, Cdk11 polypeptide production from a cell's endogenous Cdk11 gene involves first using recombination to place a recombination sequence from a site-specific recombination system (e.g., Cre/loxP, FLP/FRT) (Sauer et al., Current Opinion In Biotechnology, 5:521-527, 1994; Sauer et al., Methods In Enzymology, 225:890-900, 1993) upstream (that is, 5′ to) of the cell's endogenous genomic Cdk11 polypeptide coding region. A plasmid containing a recombination site to the site that was placed just upstream of the genomic Cdk11 polypeptide coding region is introduced into the modified cell line along with the appropriate recombinase enzyme. This recombinase causes the plasmid to integrate, via the plasmid's recombination site, into the recombination site located just upstream of the genomic Cdk11 polypeptide coding region in the cell line (Baubonis and Sauer, Nucleic Acids Res., 21:2025-2029, 1993; O'Gorman et al., Science, 251:1351-1355, 1991). Any flanking sequences known to increase transcription (e.g., enhancer/promoter, intron, translational enhancer), if properly positioned in this plasmid, would integrate in such a manner as to create a new or modified transcriptional unit resulting in de novo or increased Cdk11 polypeptide production from the cell's endogenous Cdk11 gene.

A further method to use the cell line in which the site specific recombination sequence had been placed just upstream of the cell's endogenous genomic Cdk11 polypeptide coding region is to use recombination to introduce a second recombination site elsewhere in the cell line's genome. The appropriate recombinase enzyme is then introduced into the two-recombination-site cell line, causing a recombination event (deletion, inversion, translocation) (Sauer et al., Current Opinion In Biotechnology, supra, 1994; Sauer, Methods In Enzymology, supra, 1993) that would create a new or modified transcriptional unit resulting in de novo or increased Cdk11 polypeptide production from the cell's endogenous Cdk11 gene.

An additional approach for increasing, or causing, the expression of Cdk11 polypeptide from a cell's endogenous Cdk11 gene involves increasing, or causing, the expression of a gene or genes (e.g., transcription factors) and/or decreasing the expression of a gene or genes (e.g., transcriptional repressors) in a manner which results in de novo or increased Cdk11 polypeptide production from the cell's endogenous Cdk11 gene. This method includes the introduction of a non-naturally occurring polypeptide (e.g., a polypeptide comprising a site specific DNA binding domain fused to a transcriptional factor domain) into the cell such that de novo or increased Cdk11 polypeptide production from the cell's endogenous Cdk11 gene results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is to the preselected site in the cellular chromosomal DNA with which recombination is to occur. In the construct, the exon is generally 3′ of the regulatory sequence and the splice-donor site is 3′ of the exon.

If the sequence of a particular gene is known, such as the nucleic acid sequence of Cdk11 polypeptide presented herein, a piece of DNA that is complementary to a selected region of the gene can be synthesized or otherwise obtained, such as by appropriate restriction of the native DNA at specific recognition sites bounding the region of interest. This piece serves as a targeting sequence(s) upon insertion into the cell in that it will hybridize to its region within the genome. It is conventionally believed that if this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a Cdk11 polypeptide, which nucleotides may be used as targeting sequences.

Cdk11 polypeptide cell therapy, e.g., the implantation of cells producing Cdk11 polypeptides, is also contemplated. This embodiment involves implanting cells capable of synthesizing and secreting a biologically active from of Cdk11 polypeptide. Such Cdk 11 polypeptide-producing cells can be cells that are natural producers of Cdk 11 polypeptides or may be recombinant cells whose ability to produce Cdk11 polypeptides has been augmented by transformation with a gene encoding the desired Cdk11 polypeptide or with a gene augmenting the expression of Cdk11 polypeptide. Such a modification may be accomplished by means of a vector suitable for delivering the gene as well as promoting its expression and secretion. In order to minimize a potential immunological reaction in patients being administered a Cdk11 polypeptide, as may occur with the administration of a polypeptide of a foreign species, it is preferred that the natural cells producing Cdk11 polypeptide be of human origin and produce human Cdk 11 polypeptide. Likewise, it is preferred that the recombinant cells producing Cdk11 polypeptide be transformed with an expression vector containing a gene encoding a human Cdk11 polypeptide.

Implanted cells may be encapsulated to avoid the infiltration of surrounding tissue. Human or non-human animal cells may be implanted in patients in biocompatible, semipermeable polymeric enclosures or membranes that allow the release of Cdk11 polypeptide, but prevent the destruction of the cells by the patient's immune system or by other detrimental factors from the surrounding tissue. Alternatively, the patient's own cells, transformed to produce Cdk11 polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (WO95/05452; PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for delivery of the molecules from living cells to specific sites within a recipient. In addition, see U.S. Pat. Nos. 4,892,538, 5,011,472, and 5,106,627. A system for encapsulating living cells is described in PCT Application no. PCT/US91/00157 of Aebischer et al. See also, PCT Application No. PCT/US91/00155 of Aebischer et al., Winn et al., Exper. Neurol., 113: 322-329, 1991, Aebischer et al., Exper. Neurol., 111:269-275, 1991; and Tresco et al. ASAIO, 38:17-23, 1992.

In vivo and in vitro gene therapy delivery of Cdk11 polypeptides is also envisioned. One example of a gene therapy technique is to use the Cdk11 gene (either genomic DNA, cDNA, and/or synthetic DNA) encoding a Cdk 11 polypeptide which may be operably linked to a constitutive or inducible promoter to form a “gene therapy DNA construct”. The promoter may be or heterologous to the endogenous Cdk11 gene, provided that it is active in the cell or tissue type into which the construct will be inserted. Other components of the gene therapy DNA construct may optionally include, DNA molecules designed for site-specific integration (e.g., endogenous sequences useful for recombination), tissue-specific promoter, enhancer(s) or silence(s), DNA molecules capable of providing a selective advantage over the parent cell, DNA molecules useful as labels to identify transformed cells, negative selection systems, cell specific binding agents (as, for example, for cell targeting), cell-specific internalization factors, and transcription factors to enhance expression by a vector as well as factors to enable vector manufacture.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain unintegrated.

In yet other embodiments, regulatory elements can be included for the controlled expression of the Cdk11 gene in the target cell. Such elements are turned on in response to an appropriate effector. In this way, a therapeutic polypeptide can be expressed when desired. One conventional control means involves the use of small molecule dimerizers or rapalogs (as described in WO 96/41865 (PCT/US96/099486); WO 97/31898 (PCT/US97/03137) and WO 97/31899 (PCT/US95/03157) used to dimerize chimeric proteins which contain a small molecule-binding domain and a domain capable of initiating biological process, such as a DNA-binding protein or transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain which results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See, Aridor and Balch, Science 287:816-817, 2000 and Rivera et al., Science 287: 826-830 (2000).

Other suitable control means or gene switches include, but are not limited to, the following systems. Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligan-binding domain is modified to eliminate the ability of the receptor to bind to the natural ligand. The modified steroid hormone receptor system is further described in U.S. Pat. No. 5,364,791; WO 96/40911; and WO 97/10337.

Yet another control system uses ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation domain/DNA-binding domain/ligand-binding domain to initiate transcription. The ecdysone system is further described in U.S. Pat. Nos. 5,514,578; WO9738117; WO 96/37609; and WO 93/03162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758; 5,650,298 and 5,654,168.

Additional expression control systems and nucleic acid constructs are described in U.S. Pat. Nos. 5,741,679 and 5,834,186, to Innovir Laboratories Inc.

In vivo gene therapy may be accomplished by introducing the gene encoding a Cdk11 polypeptide into cells via local injection of a Cdk11 nucleic acid molecule or by other appropriate viral or non-viral delivery vectors. (Hefti, Neurobiology, 25:1418-1435, 1994). For example, a nucleic acid molecule encoding a Cdk11 polypeptide may be contained in an adeno-associated virus (AAV) vector for delivery to the targeted cells (e.g., Johnson, International Publication No. WO95/34670; International Application No. PCT/US95/07178). The recombinant AAV genome typically contains AAV inverted terminal repeats flanking a DNA sequence encoding a Cdk11 polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

It is also contemplated that Cdk11 gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A means to increase endogenous Cdk11 polypeptide expression in a cell via gene therapy is to insert one or more enhancer element(s) into the promoter of the Cdk11 gene, where the enhancer element(s) can serve to increase transcriptional activity of the Cdk11 gene. The enhancer element(s) used will be selected based on the tissue in which one desires to activate the gene(s); enhancer elements known to confer promoter activation in that tissue will be selected. For example, if a gene encoding a Cdk11 polypeptide is to be “turned on” in T-cells, the Ick promoter enhancer element may be used. Here, the functional portion of the transcriptional element to be added may be inserted into a fragment of DNA containing the cdk11 polypeptide promoter Sand optionally, inserted into a vector and/or 5′ and/or 3′ flanking sequence(s), etc.) using standard cloning techniques. This construct, known as a “recombination construct”, can then be introduced into the desired cells either ex vivo or in vivo.

Gene therapy also can be used to decrease Cdk11 polypeptide expression by modifying the nucleotide sequence of the endogenous promoter(s). Such modification is typically accomplished via recombination methods. For example, a DNA molecule containing all or a portion of the promoter of the Cdk11 gene(s) selected for inactivation can be engineered to remove and/or replace pieces of the promoter that regulate transcription. For example the TATA box and/or the binding site of a transcriptional activator of the promoter may be deleted using standard molecular biology techniques; such deletion can inhibit promoter activity thereby repressing the transcription of the corresponding Cdk11 gene. The deletion of the TATA box or the transcription activator binding site in the promoter may be accomplished by generating a DNA construct comprising all or the relevant portion of the Cdk11 polypeptide promoter(s) (from the same or a related species as the Cdk11-gene(s) to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. The construct will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via recombination, where the 5′ and 3′ DNA sequences in the promoter construct can serve to help integrate the modified promoter region via hybridization to the endogenous chromosomal DNA.

Additional Uses of Cdk11 Nucleic Acids and Polypeptides

Nucleic acid molecules of the present invention (including those that do not themselves encode biologically active polypeptides) may be used to map the locations of the Cdk 11 gene and related genes on chromosomes. Mapping may be done by techniques known in the art, such as PCR amplification and in situ hybridization.

Cdk11 nucleic acid molecules (including those that do not themselves encode biologically active polypeptides), may be useful as hybridization probes in diagnostic assays to test, either qualitatively or quantitatively, for the presence of a Cdk11 DNA or corresponding RNA in mammalian tissue or bodily fluid samples. Cdk11 may serve as a diagnoses/prognosis marker or assay for a wide variety of human cancers. Monitoring changes in the expression of Cdk11 during cancer treatment may be used as a surrogate marker to monitor tumor growth and treatment success.

The Cdk11 polypeptides may be used (simultaneously or sequentially) in combination with one or more cytokines, growth factors, antibiotics, anti-inflammatories, and/or chemotherapeutic agents as is appropriate for the indication being treated. Cdk11 may be useful as a small molecule inhibitor. In addition, peptide inhibitors designed from Cdk11 polypeptide may be used as a therapeutic or identifying substance which modulates Cdk11 polypeptide activity.

Other methods may also be employed where it is desirable to inhibit the activity of one or more Cdk11 polypeptides. Such inhibition may be effected by nucleic acid molecules which are complementary to and hybridize to expression control sequences (triple helix formation) or to Cdk 11 mRNA. For example, antisense DNA or RNA molecules, which have a sequence that is complementary to at least a portion of the selected Cdk11 gene(s) can be introduced into the cell. Antisense probes may be designed by available techniques using the sequence of Cdk11 polypeptide disclosed herein. Typically, each such antisense molecule will be complementary to the start site (5′ end) of each selected Cdk11 gene. When the antisense molecule then hybridizes to the corresponding Cdk11 mRNA, translation of this mRNA is prevented or reduced. Antisense inhibitors provide information relating to the decrease or absence of a Cdk11 polypeptide in a cell or organism.

Alternatively, gene therapy may be employed to create a dominant-negative inhibitor of one or more Cdk11 polypeptides. In this situation, the DNA encoding a mutant polypeptide of each selected Cdk11 polypeptide can be prepared and introduced into the cells of a patient using either viral or non-viral methods as described herein. Each such mutant is typically designed to compete with endogenous polypeptide in its biological role. Particularly, Cdk11 contains a kinase domain that may be useful in designing dominant negative gene therapy for treatment in a wide variety of tumors

In addition, a Cdk11 polypeptide, whether biologically active or not, may be used as an immunogen, that is, the polypeptide contains at least one epitope to which antibodies may be raised. Selective binding agents that bind to a Cdk11 polypeptide (as described herein) may be used for in vivo and in vitro diagnostic purposes, including, but not limited to, use in labeled form to detect the presence of Cdk11 polypeptide in a body fluid or cell sample. The antibodies may also be used to prevent, treat, or diagnose a number of diseases and disorders, including those recited herein. The antibodies may bind to a Cdk 11 polypeptide so as to diminish or block at least one activity characteristic of a Cdk11 polypeptide, or may bind to a polypeptide to increase at least one activity characteristic of a Cdk11 polypeptide (including by increasing the pharmacokinetics of the Cdk11 polypeptide).

cDNA encoding Cdk11 polypeptide in E. coli strain INVα-F was deposited with the ATCC on Nov. 3, 2000 and has Accession No. PTA-2649.

The following examples are intended for illustration purposes only, and should not be construed as limiting the scope of the invention in any way.

### EXAMPLE 1

### Cloning of Human Cdk11

Materials and methods for cDNA cloning and analysis are described in Sambrook et al., supra.

A search was performed in a proprietary database to identify EST sequences with homology to Cdk7. This search identified a rat EST (MRPE3-00072-A7-Z; SEQ ID NO: 5) as potentially encoding a polypeptide exhibiting 45% homology to Cdk7. The EST sequence was used to search the public domain (Genbank search) which identified a human protein (Genbank accession no. AF035013; SEQ ID NO: 8) which was 82% identical to the EST sequence (SEQ ID NO: 5).

Polymerase chain reaction (PCR) primers were designed to flank the deduced coding region of the identified Genbank nucleotide sequence (SEQ ID NO: 7). The primer sequences were as follows: 5′ primer: GGGGTGGAGGAGAAGTGGAGT (SEQ ID NO: 3) and 3′ primer: GGCTCTGAGAATAAAaCCAAACCAA (SEQ ID NO: 4). These primers were used to amplify the coding region from four independent human Marathon cDNA libraries (Clontech, Palo Alto, Calif.) from fetus, brain, testis and pancreas. The nucleotide sequence of the amplified coding regions were determined using standard sequencing methods (See Sambrook et al., supra.). Multiple independent sequences derived from each of the tissues identified errors in the published Genbank nucleotide and predicted amino acid sequences (SEQ ID NOS: 7 and 8, respectively).

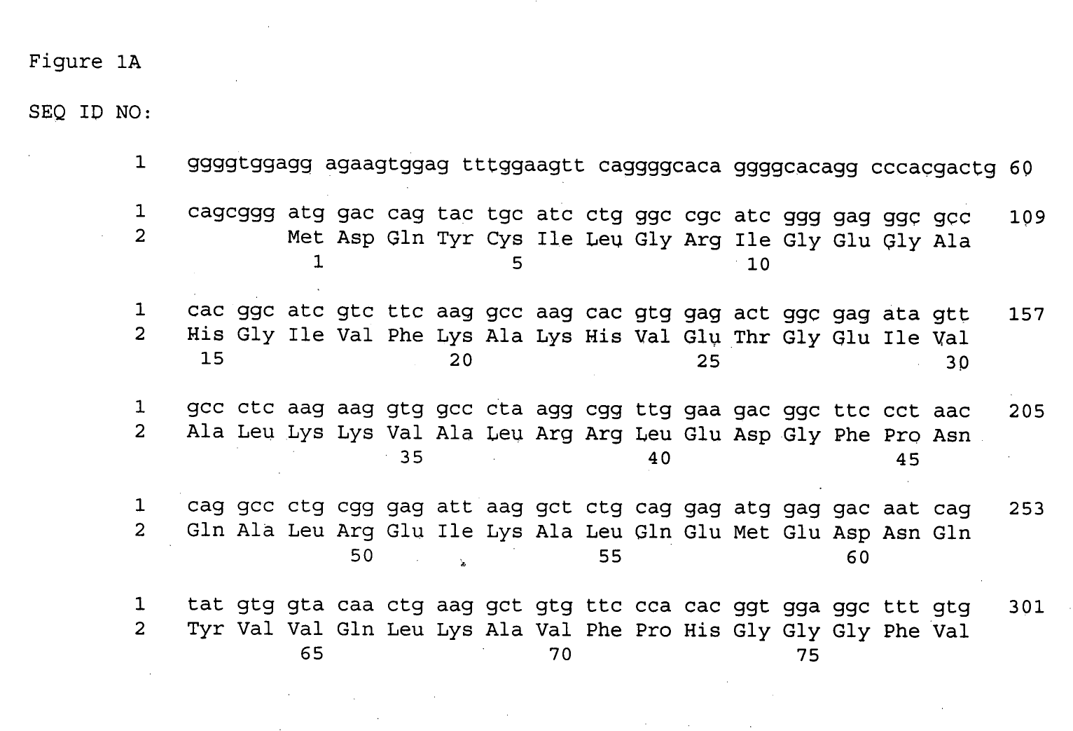

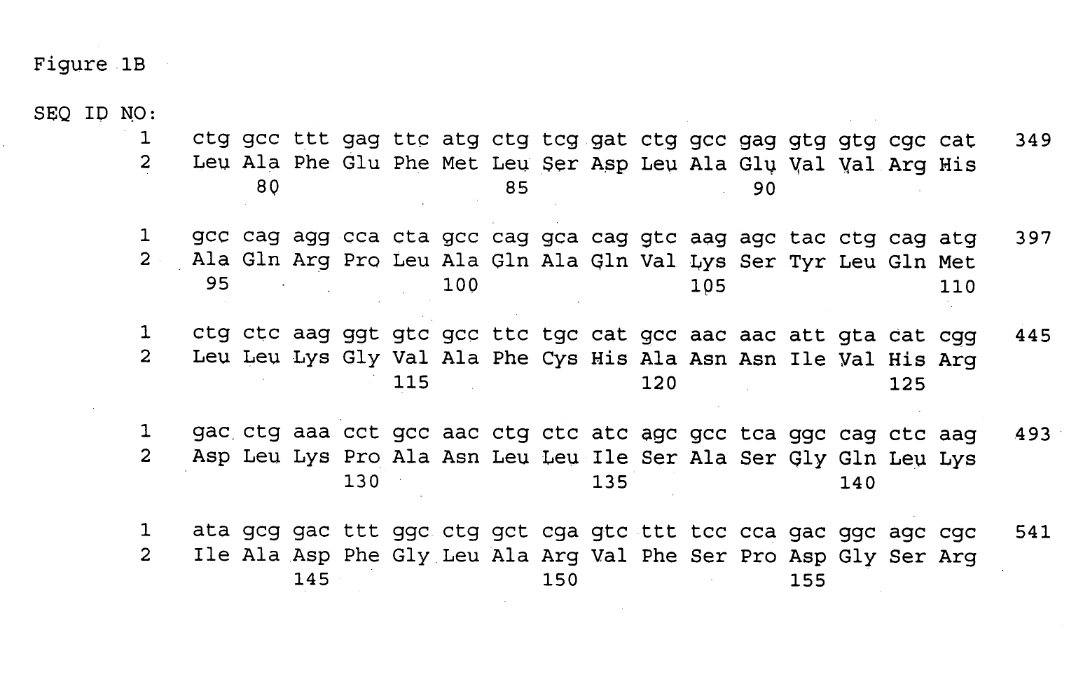

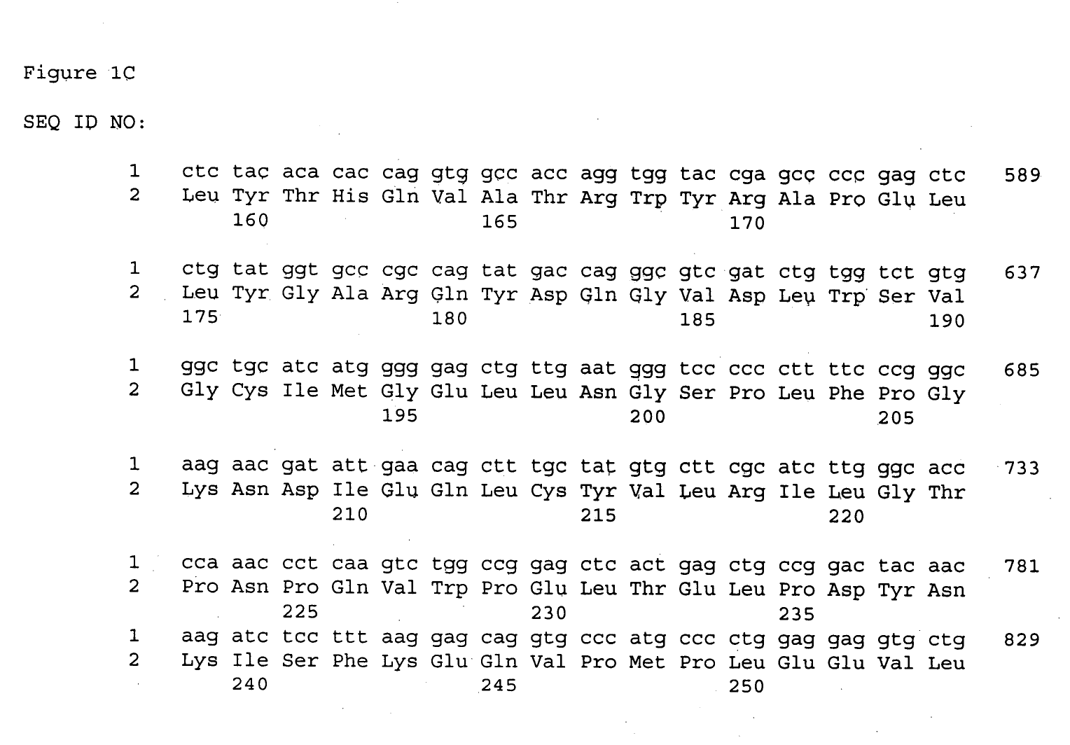

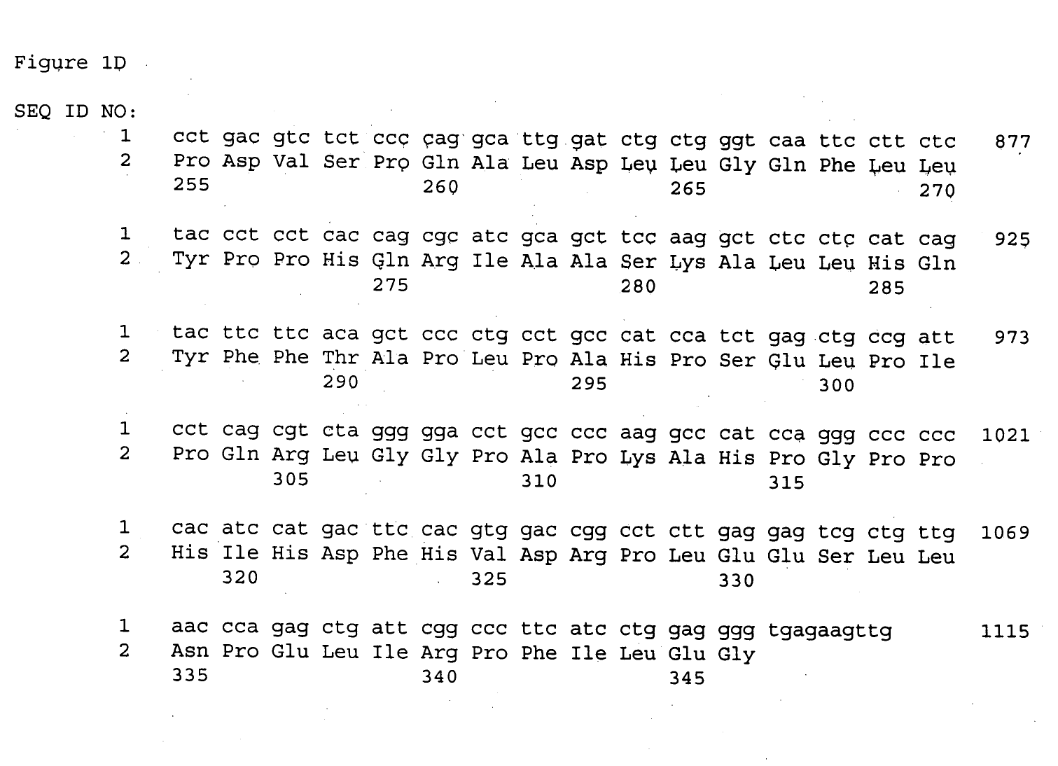

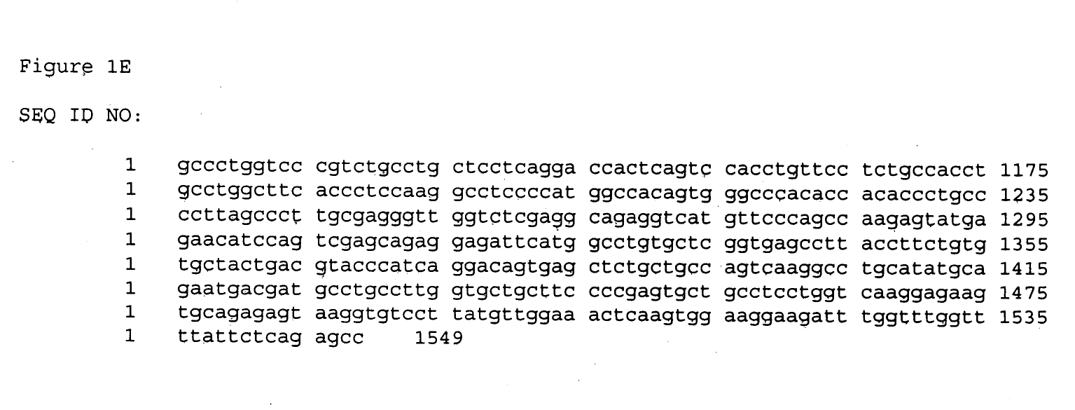

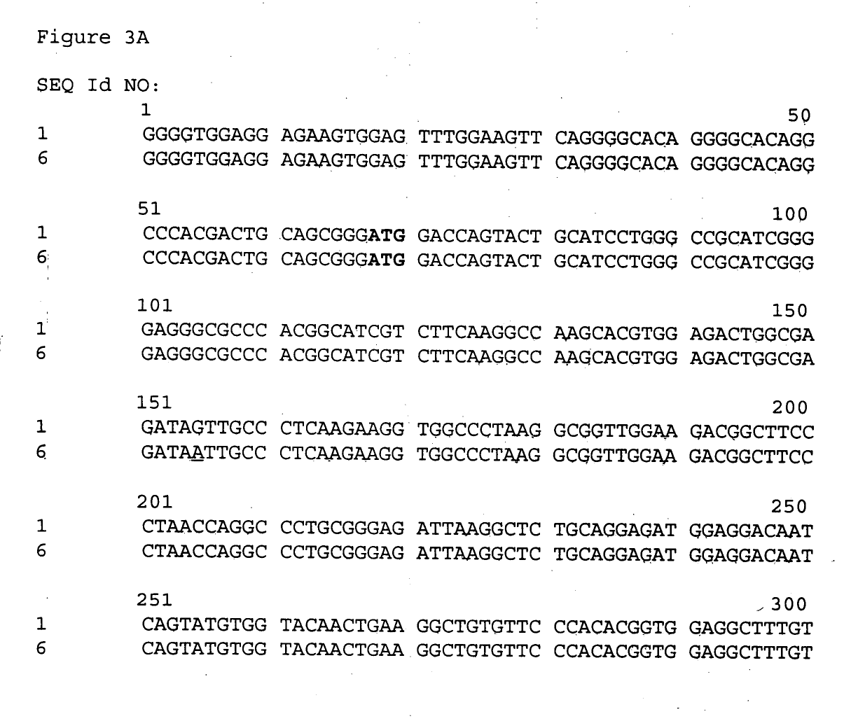

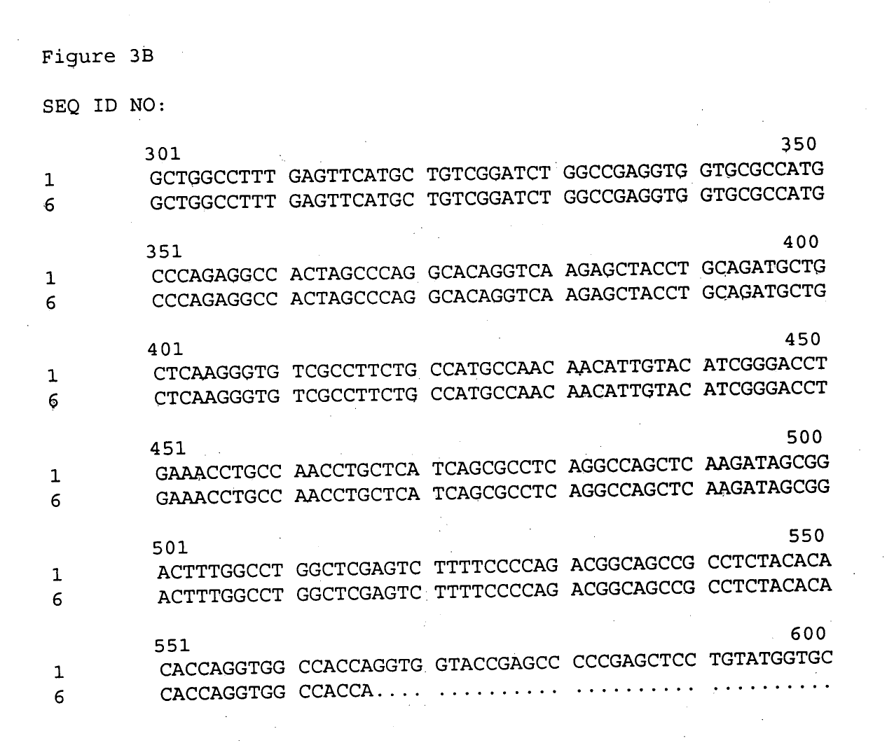

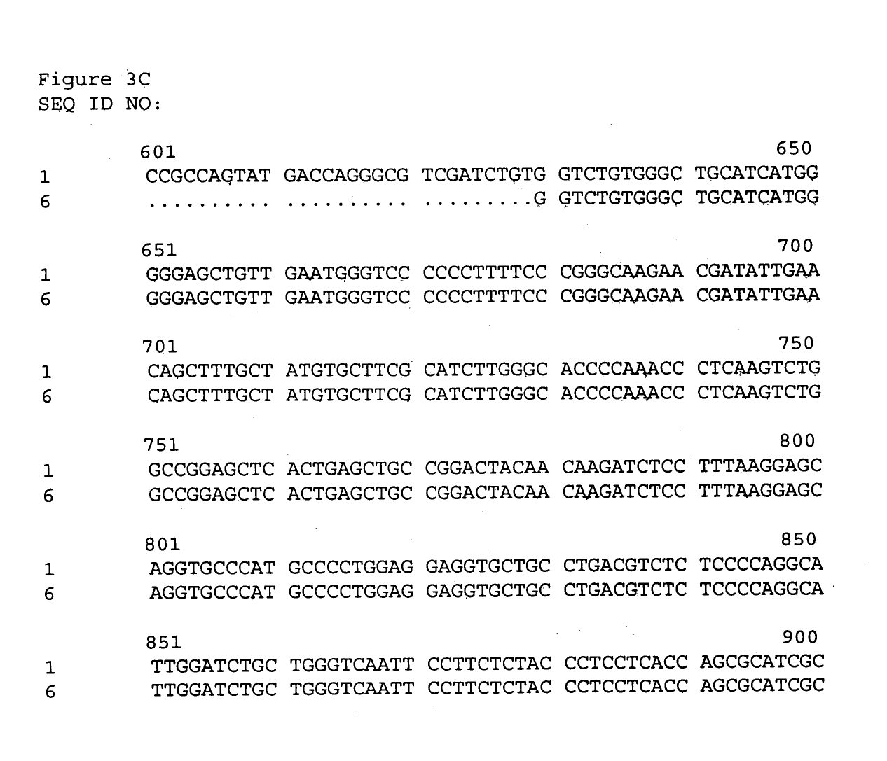

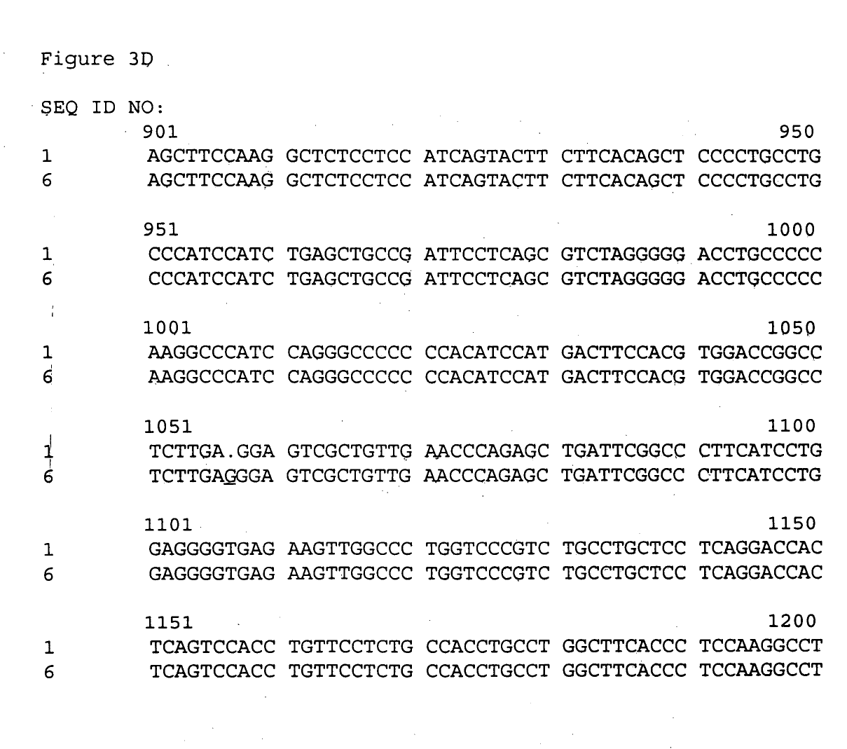

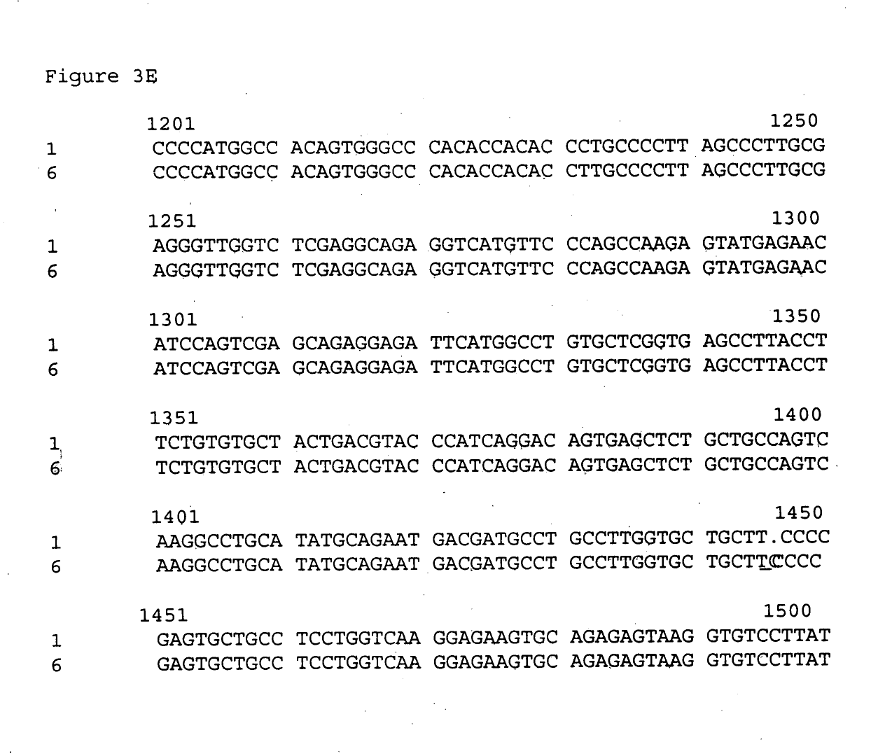

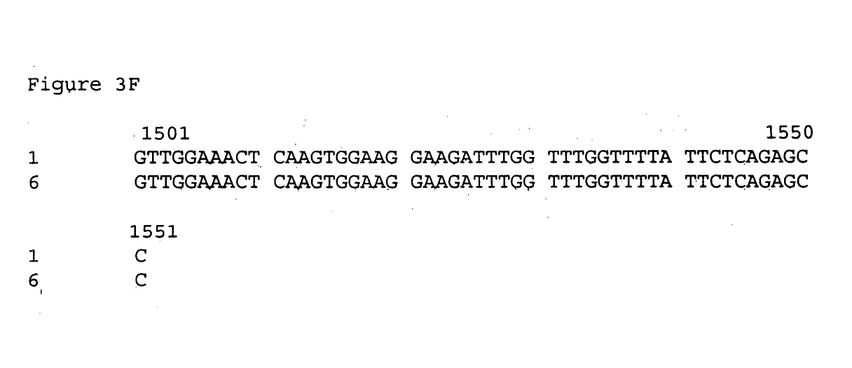

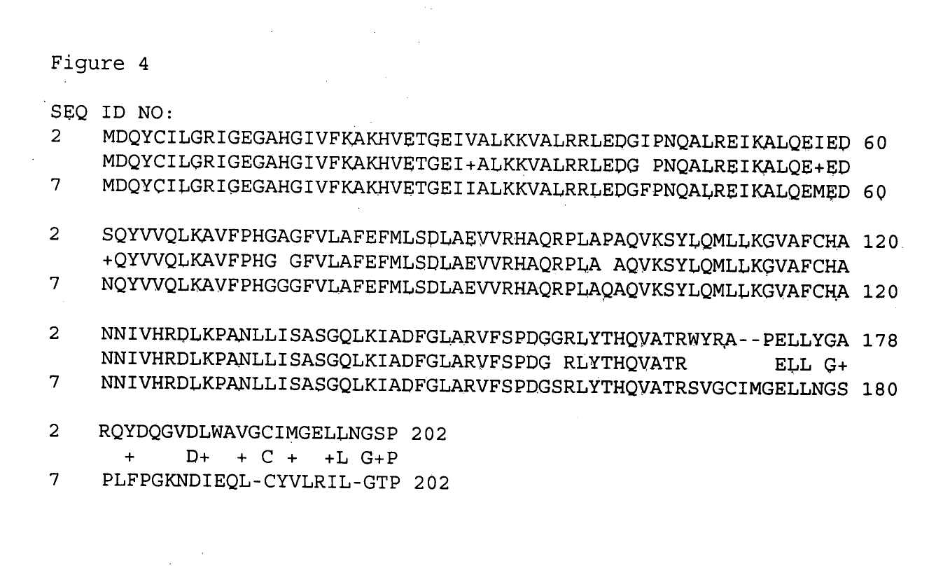

The polynucleotide sequence identified in the present cloning procedure is set out as SEQ ID NO: 1, and denoted herein as Cdk11. As displayed in FIG. 1, the Cdk11 polynucleotide is 1550 nucleotides in length and with a coding region of 1038 nucleotides that encodes a 346 amino acid protein (SEQ ID NO: 2). FIG. 3 displays the differences in the nucleotide sequence of Cdk11 (SEQ ID NO: 1) and the Genbank sequence (SEQ ID NO: 7), while FIG. 4 displays the differences of the predicted amino acid sequence of Cdk11 (SEQ ID NO: 2) and predicted amino acid sequence based on the Genbank sequence (SEQ ID NO: 8). The Genbank polynucleotide sequence incorrectly predicts a 452 amino acid polypeptide due to a frameshift mutation at nucleotide 1227 where an additional guanine, not present in any of the cloned sequences of the present invention, exists. Additionally, the Genbank sequence contained a 63 nucleotide deletion resulting in the absence of nucleotides 168-188 found in Cdk11 (SEQ ID NO: 1). The inframe deletion was also found in one of the five Cdk11 clones isolated from the human brain library suggesting that this may represent a genuine Cdk11 splice variant. The Genbank amino acid sequence (SEQ ID NO: 8) also contained a missense mutation, as compared to the Cdk11 amino acid sequence (SEQ ID NO: 2), which changed the valine at position 30 to an isoleucine.

The regions that appeared to lack a strong consensus between the originally identified rat EST and the human Genbank sequence were due to the errors described above. The Cdk11 sequence (SEQ ID NO: 1) characterized encodes a polypeptide that is identical in length and is 93% identical in sequence to the rat EST polypeptide sequence (SEQ ID NO: 6) as shown in FIG. 2. Thus, the rat EST (SEQ ID NO: 5) encodes the full length rat Cdk11 polypeptide-sequence.

### EXAMPLE 2

### Evaluation of Tissue Expression of Cdk11

A. Northern Blot and Dot Blot Analysis

Cdk11 tissue expression was evaluated by Northern and Dot blot analysis. See Sambrook et al., supra. A full length Cdk11 DNA probe was generated by performing PCR on a human fetus Marathon cDNA library (Clontech) using the Advantage cDNA PCR kit (Clontech) according to the manufacturer's instructions. PCR was carried out for 25 cycles of 94° C. for 30 seconds, 65° C. for 30 seconds and 72° C. for 2 minutes with the following primers:: 5′ primer: ATGGACCAGTACTGCATCCTGGGCCGCATC (SEQ ID NO: 9) 3′ primer: ACCTTACTCTCTGCACTTCTCCTTGAC (SEQ ID NO: 10). The full length probe was ³²P-labeled using a random labeling kit (Ambion, Austin, Tex.; cat. no. 1470) according to the manufacture's instructions.

Dot blot analysis was performed on preblotted membranes containing multiple human tissues available from Clontech (RNA Master Blot, cat. no. 7770-1). Northern blot analysis was also performed on a multiple human tissue blots also available from Clontech (Human I cat no. 7760-1; Human II cat. no. 7767-1) and a preblotted membrane containing RNA from various human cancer cell lines (Clontech; cat. no. 7757-1). The blots were prehybridized in buffer containing 5×SSC, 50% formamide, 2.5×Denhardt's solution, 0.5% SDS and 100 μg of sheared salmon sperm DNA for 2 hours at 42° C. The blots were then hybridized in the above-describe buffer containing about 50,000 cpm/ml (as quantitated in a scintillation counter) of ³²P-labeled probe overnight at 42° C. The blots were washed 2 times under high stringency conditions (0.2×SSC/0.5% SDS) for 30 minutes at 55° C. Radioactive blots were exposed to X-ray film for 3 days at −80° C. under intensifying screens. After development, the results were quantitated using NIH Image 1.60.

The RNA Master Blot contained mRNA from the following tissues: ovary, testis, stomach, prostate, uterus, bladder, colon, skeletal muscle, aorta, heart, spinal cord, acumens, thalamus, temporal lobe, substantia nigra, putamen, occipital lobe, medulla oblongata, hippocampus, frontal lobe, cerebral cortex, cerebellum, caudata nucleus, amygdala, whole brain, fetal lung, fetal thymus, fetal spleen, fetal kidney, fetal liver, fetal heart, fetal brain, placenta, trachea, lung, bone marrow, lymph node, peripheral leukocytes, thymus, spleen, small intestine, appendix, liver, kidney, mammary gland, thyroid gland, adrenal gland, pituitary gland and pancreas.

Dot blot analysis revealed that Cdk11 was expressed at high levels (greater than 10 expression units) in testis, amygdala, whole brain, fetal lung, fetal kidney, fetal brain, and kidney. The analysis also revealed that Cdk11 was not detected in colon, skeletal muscle, aorta, heart, bone marrow, lymph node peripheral leukocytes and spleen.

The multiple tissue Northern blots contained mRNA from the following tissues: pancreas, kidney, skeletal muscle, liver, lung, placenta, brain, heart, peripheral blood leukocytes, colon, small intestine, ovary, testis, prostate, thymus and spleen. Northern blot analysis indicated Cdk11 was expressed at high level in testis tissue and was undetectable in liver, lung, skeletal muscle, placenta, peripheral blood leukocytes and thymus.

The human cancer cell line blot contained mRNA from the following tumor cell lines: G631, A549, SW480, RAJ1, MOLT4, K562, Hela 53 and HL-60. Northern blot analysis indicated that Cdk11 was expressed in all cell lines except G631 and RAJ1 with high expression (≧10 expression units) in A549, MOLT4 and K562.

B. Quantitative PCR Analysis

Cdk11 expression levels in tumor and normal tissues samples, obtained from Biochain, were compared using quantitative PCR analysis (Taqman™ analysis). The PCR reaction consisted of the Taqman™ One-Step RT-PCR Master Mix Reaction Kit (Applied Biosystems), 25 ng tumor or normal tissue, the 5′ primer (TGCCAACCTGCTGATCAGC; SEQ ID NO: 11), the 3′ primer (AGACTCGAGCCAGGCCAAA; SEQ ID NO: 12) and a probe (CCTCAGGCCAGCTCAAGATAGCA; SEQ ID NO: 13) linked to a reporter dye (6-fam) at the 5′ end and linked to a quencher dye (Tamara) at the 3′ end. The PCR reaction was carried out in a ABI PRISM 7700 Sequence Detector (Applied Biosystems) under the following cycling conditions: 48° C. for 2 minutes, 48° C. for 30 minutes, 95° C. for 10 minutes then 40 cycles of 95° C. for 15 minutes and 60° C. for 1 minute. The results were normalized to the S9 ribosomal gene and the values were compared to cyclin A expression levels in the same tissue samples.

The following tumor tissues and corresponding normal tissues were obtained from Biochain (Hayward, Calif.): lung, breast (1), colon, rectum, liver, ovary, prostate, breast (2), and uterus. Table III provides a description of the tumor and corresponding normal tissues.

TABLE III

| Tissue | | Lot No. | Sex | Age | Comments |
|---|---|---|---|---|---|
| Lung | Normal | A208043 | Male | 57 | |
| | Tumor | A208048 | Female | 43 | Poorly differentiated |
| | | | | | squamous cell/ |
| | | | | | Bronchiola alveolar |
| | | | | | cell carcinoma |
| Breast 1 | Normal | A207160 | Female | 54 | |
| | Tumor | A208060 | Female | 79 | Well differentiated |
| | | | | | invasive ductal |
| | | | | | carcinoma |
| Colon | Normal | A208041 | Male | 27 | |
| | Tumor | A208047 | Male | 68 | Well differentiated |
| | | | | | adenocarcinoma |
| Rectum | Normal | A206056 | Male | 26 | |
| | Tumor | A210034 | Female | 61 | Well differentiated |
| | | | | | adenocarcinoma |
| Liver | Normal | A205002 | Male | 26 | |
| | Tumor | A210011 | Male | 53 | Moderately differentiated |
| | | | | | hepatocellular carcinoma |
| Ovary | Normal | A208038 | Female | 47 | |
| | Tumor | A209083 | Female | 49 | Poorly differentiated |
| | | | | | carcinoma |
| Prostate | Normal | A302133 | Male | 25 | |
| | Tumor | A209134 | Male | 68 | Hyperplasia |
| Breast 2 | Normal | A207171 | Female | 53 | |
| | Tumor | A206035 | Female | 48 | Moderately differentiated |
| | | | | | invasive ductal |
| | | | | | carcinoma |
| Uterus | Normal | A206035 | Female | 39 | |
| | Tumor | A209094 | Female | 41 | Leionyima |

Cdk 11 expression was elevated in breast (1), breast (2), ovary and uterus tumor tissue as compared to normal tissue. These same tissue samples (except for breast (2)) also had elevated expression levels of cyclin A.

### EXAMPLE 3

### Cdk11 Expression Profile

Quantitative PCR analysis was used to determine the cell cycle expression profile of Cdk11 in restimulated human fibroblasts as compared to the cell cycle expression profiles of Cdk2, Cyclin E1 and Cyclin E2. The quantitative PCR reaction was carried out (as described in Example 2) on RNA samples taken from serum restimulated human foreskin fibroblast (ATCC No. CRL 2091). The fibroblasts were cultured in the absence of fetal bovine serum (FBS) then restimulated with 10% FBS as described in Lyer et al., Science, 283: 83-87, 1999. The RNA samples were collected at various times points post-restimulation (0, 1.5, 4, 6, 8, 13, 15, 21 and 24 hours). The values obtained from the analysis were normalized to the expression of the S9 ribosomal gene transcript. The primer sequences used for the quantitative PCR for Cdk 11 are set out in Example 2 (SEQ ID NOS: 11 and 12) and the others are set out in Table IV.

TABLE IV

| | | | |
|---|---|---|---|
| Cdk2 | 5′primer | GGTGGTGTGGCCAGGAGTT | SEQ ID NO: 20 |
| | 3′primer | TCTTGCCGGGCCCACT | SEQ ID NO: 21 |
| | Probe | TTCTATGCCTGATTACAAG | SEQ ID NO: 22 |
| | | CCAAGTTTCCCC | |
| Cyclin E1 | 5′primer | AACGTGCAAGCCTCGGATT | SEQ ID NO: 23 |
| | 3′primer | GCCCAGCTCAGTACAGGCA | SEQ ID NO: 24 |
| | Probe | TTGCACCATCCAGAGGCTC | SEQ ID NO: 25 |
| | | CCC | |
| Cyclin E2 | 5′primer | CATTAGAGTTCCAGTACAG | SEQ ID NO: 26 |
| | | AATACTGACTG | |
| | 3′primer | ACCTGAGGCTTTCTTAACC | SEQ ID NO: 27 |
| | | ACTTC | |
| | Probe | TGCTGCCTTGTGCCATTTT | SEQ ID NO: 28 |
| | | ACCTCCAT | |

Cdk11 expression levels did not appear to significantly change during progression through the cell cycle. In comparison to expression of genes known to change during cell cycle progression (cyclin E1 and E2), Cdk11 expression levels were low and remained at relatively base line level throughout cell cycle progression.

### EXAMPLE 4

### Production of Anti-Cdk11 Polypeptide Antibodies

Polyclonal antibodies were generated against a Cdk11 peptide produced by chemical synthesis. Suitable procedures for generating antibodies include those described in Hudson and Hay, Practical Immunology, 2nd Edition, Blackwell Scientific Publications (1980).

Rabbits were injected with the Cdk11 peptide corresponding to amino acids 311-327 of SEQ ID NO: 2 (CPKAHPGPPHIHDFHVDR; SEQ ID NO: 14). The peptide was coupled to keyhole limpet hemocyanin (KLH) using disuccinimidyl suberate as a cross linker. To facilitate conjugation to KHL, a carboxy-terminal cysteine was added to the Cdk11 peptide sequence using methods known in the art. The sera was collected from the immunized rabbits and affinity purified with a Sulfolink resin that contained the peptide couple via the amino terminal Cys.

### EXAMPLE 5

### Production of Cdk11 Polypeptides

A. Mammalian Cell Production

Cdk11 was amplified and subcloned utilizing primers corresponding to the 5′ and 3′ ends of the coding sequence. The primer sequences were designed to introduce a 5′ NotI site and a 3′ EcoRI site and either an amino terminal or a carboxy terminal FLAG sequence. For the N-terminal FLAG clone the primers were as follows: 5′ primer TGCGGCCGCATGGACTACAAAGACGAT GACGACAAGATGGACCAGTACTGCATCCTG and 3′ primer: GAATTCTCACCCCTCCAGGATGAAGGG (SEQ-ID NOS: 29 and 30, respectively). For the C-terminal FLAG clone the primers were as follows: 5′ TGCGGCCGCATGGACCAGTACTGCATCCTG and 3′ primer GAATTCTCACTTGTCGTCATCGTCTTTGTAGTCCCCCTCCAGGATGAAGGG (SEQ ID NO: 31 and 32, respectively). PCR products were gel purified and inserted into the pcDNA3.1 expression vector (Invitrogen) using standard recombinant DNA techniques. The pcDNA3.1 vector containing the Cdk11 insert was transiently transfected into 293T cells (ATTC NO. CRL-1573) using calcium phosphate mediated transfection. The cells were harvested in SDS sample buffer and analyzed by SDS-PAGE. (See Sambrook et al., supra.)

Cdk11 polypeptide was produced as a fusion protein with a FLAG epitope tag which was detected by Western blot analysis using anti-FLAG antibodies (Kodak) and anti-Cdk 11 polyclonal antibodies (generated as described in Example 3).

B. Insect Cell Production

Recombinant bacmid DNA was generated using the Bac-to-Bac system (Life Technologies, Rockville, Md.) according to the manufacturer's instructions. Recombinant bacmid DNA was then transfected into Sf9 cells (ATCC No. CRL-1711). After approximately 60 hours of incubation, the media containing recombinant baculovirus particles was harvested (passage 0 virus), and the remaining cells were assayed for Cdk 11 polypeptide expression as described above.

Passage 0 virus was successfully amplified to generate higher titer viral stocks. This was accomplished by infecting 1×10⁶(50 ml) adherent Sf9 cells with 0.5 ml of passage 0 virus. Subsequently, the high titer baculovirus stock was used to infect Sf9 and Hi5 cells. In general, 1 ml of passage 2 or passage 3 virus was used to infect 2×10⁸ cells. The infected cells were incubated for 48 to 60 hours and harvested. Untagged Cdk11 polypeptides were produced and detected by Western blot with Cdk11 antibodies (generated as described in Example 3).

Alternatively, Cdk11 polypeptide could be produced as a fusion protein with a 6×HIS epitope tag which is detected by Western blot analysis using anti-HIS antibodies (Pentahis Antibody; Qiagen). This recombinant Cdk11 polypeptide could then be purified with a Nickel affinity resin wherein the 6× His tag will bind to the nickel resin.

### EXAMPLE 6

### Monoclonal Antibody Production

For the production of monoclonal antibodies, animals (typically mice or rabbits) are injected with a Cdk11 antigen (such as an Cdk11 polypeptide), and those with sufficient serum titer levels as determined by ELISA are selected for hybridoma production. Spleens of immunized animals are collected and prepared as single-cell suspensions from which splenocytes are recovered. The splenocytes are fused to mouse myeloma cells (such as Sp2/0-Ag14 cells; ATCC no. CRL-1581), allowed to incubate in DMEM with 200 U/ml penicillin, 200 mg/ml streptomycin sulfate, and 4 mM glutamine, then incubated in HAT selection medium (Hypoxanthine; Aminopterin; Thymidine). After selection, the tissue culture supernatants are taken from each fusion well and tested for anti-Cdk11 antibody production by ELISA. (See Kohler and Milstein, Nature 256: 495-497, 1975).

Alternative procedures for obtaining anti-Cdk11 antibodies may also be employed, such as the immunization of transgenic mice harboring human Ig loci for the production of human antibodies, and the screening of synthetic antibody libraries, such as those generated by mutagenesis of an antibody variable domain. See Hudson and Hay, Practical Immunology, 2nd Edition, Blackwell Scientific Publications (1980).

### EXAMPLE 7

### Substrate Phage Display for Enzyme Characterization of Cdk11 Polypeptide

Phage display can be used as a technology tool to improve kinetic properties of existing enzyme-substrates and/or to discover novel substrates for known, as well as orphan, enzymes. This technology, “Substrate Phage Display”, draws benefits from the ability of combinatorial phage libraries to present billions of unique peptides to an enzyme target, such as kinases, phosphatases or proteases, and allows the target enzyme to identify novel substrates that are kinetically most desirable (See Matthews and Wells, Science, 260: 1113-1117, 1993; Schmitz et al., J. Mol. Biol., 260: 664-677, 1996; Gram et al., Eur. J. Biochem., 246: 633-637, 1997). When identified, such optimum substrates can be used in high throughput screens of small molecules to discover potential drug candidates such as antagonists and agonists. Substrate Phage Display, therefore, can directly and significantly facilitate drug discovery for validated enzyme targets that do not have optimum synthetic substrates, and those novel ones for which neither substrates nor associated cellular functions are known.

Novel kinases, such as Cdk11 polypeptide, can be characterized for substrate specificity, normal cellular functions, and potential implications in disease processes by discovering optimum peptide substrates through phage display, and applying them toward identifying agents that modulate Cdk11 polypeptide biological activity. Identification of Cdk11 polypeptide as a viable drug target, and discovery of its agonists and antagonists can be accomplished by using a phage library which displays greater than 109 unique, random peptides of 7 to 30 amino acids. These peptides, within the phage display library, are exposed to Cdk11 polypeptide as potential kinase substrates. Those phages displaying phosphorylated peptides are separated by affinity-selection using anti-phosphothreonine-proline antibodies (e.g., cat no. 939915, New England Biolabs, MA). The selected phage populations are amplified and the phosphorylation-antibody selection process is repeated. Once optimal peptide substrates are identified, the cDNA in the phage genome, which encodes these peptide substrates, is sequenced and chemically synthesized with appropriate modifications (such as biotinylation). Homogenous time resolved fluorescence (HTRF) assays using biotinylated peptide substrate(s) are carried out consisting of the following components: streptavidin-allophycocyanin conjugate, Cdk11 polypeptide, and Europium-labeled anti-phosphothreonine-proline antibody (e.g., LANCE Eu—W1024 anti-pThr-Pro, from Wallac). These HTRF assays are then implemented in a high throughput format in the presence of small molecules to identify potential small molecule antagonists and agonists of Cdk11 poly.

While the present invention has been described in terms of the preferred embodiments, it is understood that variations and modifications will occur to those skilled in the art. Therefore, it is intended that the appended claims cover all such equivalent variations which come within the scope of the invention as claimed.

1-14. (canceled)

15. The process according to claim 93,
wherein the nucleic acid molecule comprising a nucleotide sequence selected from the group consisting of:
(a) the nucleotide sequence set forth in SEQ ID NO: 1
(b) a nucleotide sequence encoding the polypeptide set forth in SEQ ID NO: 2 and
(c) a nucleotide sequence which hybridizes under highly stringent conditions to the complement of (a) or (b), wherein the encoded polypeptide has an activity of the polypeptide set forth in SEQ ID NO: 2.

16-66. (canceled)

67. A method for identifying antagonists of Cdk11 biological activity comprising:
(a) contacting a small molecule compound with a Cdk11 polypeptide;
(b) detecting the biological activity of Cdk11 in the presence of said small molecule compound; and
(c) comparing the level of Cdk11 biological activity in the presence and absence of said small molecule compound.

68. The method of claim 67 wherein the small molecule compound is a member of a naturally occurring chemical library.

69. The method of claim 67 wherein the small molecule compound is a member of a naturally occurring medicinal chemical library.

70. The method of claim 67 wherein the small molecule compound is a member of a combinational chemical library.

71-89. (canceled)

90. A process for identifying a test molecule that modulates Cdk11 polypeptide activity or production, the process comprising:
(a) exposing a cell to the test molecule, wherein said cell is transformed or transfected with a nucleic acid molecule comprising a nucleotide sequence selected from the group consisting of:
(i) a nucleotide sequence encoding a polypeptide that is at least about 90% identical to the polypeptide set forth in SEQ ID NO: 2, wherein the polypeptide has an activity of the polypeptide set forth in SEQ ID NO: 2;
(ii) a nucleotide sequence encoding an allelic variant or splice variant of the nucleotide sequence set forth in SEQ ID NO: 1, wherein the encoded polypeptide has an activity of the polypeptide set forth in SEQ ID NO: 2;
(iii) a nucleotide sequence of SEQ ID NO: 1, (i), (ii), or SEQ ID NO: 1 encoding a polypeptide fragment of at least about 25 amino acid residues, wherein the polypeptide has an activity of the polypeptide set forth in SEQ ID NO: 2;
(iv) a nucleotide sequence of (i)-(iii) or SEQ TD NO: 1 comprising a fragment of at least about 16 nucleotides;
(v) a nucleotide sequence which hybridizes under highly stringent conditions to the complement of any of (a)-(d), wherein the polypeptide has an activity of the polypeptide set forth in SEQ ID NO: 2;
(b) measuring Cdk11 polypeptide activity or production in said cell, and;
(c) comparing activity of Cdk11 in cells exposed to the test molecule with activity in cells not exposed to the test molecule, thereby identifying a test molecule as a modulator of Cdk11 polypeptide activity or production.

91. The method of claim 90, wherein the test molecule is an inhibitor of Cdk11 polypeptide activity or production.

92. The method of claim 90, wherein the test molecule is a stimulator of Cdk 11 polypeptide activity or production.

93. The method of claim 90, wherein the test molecule is selected from the group consisting of a nucleic acid, a protein, a peptide, a carbohydrate, a lipid and a small molecule.

94. The method of claim 90, wherein the nucleic acid molecule comprises a nucleotide sequence at least 90% identical to the nucleotide sequence set forth in SEQ ID NO: 1.

95. The method of claim 90, wherein the nucleic acid molecule comprises a nucleotide sequence at least 95% identical to the nucleotide sequence set forth in SEQ ID NO: 1.

96. The method of claim 67, wherein the Cdk11 polypeptide comprises an amino acid sequence selected from the group consisting of:
(a) the amino acid sequence set forth in SEQ ID NO: 2 and fragments thereof that have an activity of the polypeptide set forth in SEQ ID NO: 2;
(b) the mature amino acid sequence set forth in SEQ ID NO: 2, comprising a mature amino terminus at residue 1, optionally further comprising an amino terminal methionine;
(c) an amino acid sequence for an ortholog of SEQ ID NO: 2, wherein the encoded polypeptide has an activity of the polypeptide set forth in SEQ ID NO: 2; and
(d) an amino acid sequence that is at least 90% identical to the amino acid sequence set forth in SEQ ID NO: 2, wherein the polypeptide has an activity of the polypeptide set forth in SEQ ID NO: 2.

97. The method of claim 67, wherein the polypeptide comprises an amino acid sequence at least 90% identical to the amino acid sequence set forth in SEQ ID NO: 2.

98. The method of claim 67, wherein the polypeptide comprises an amino acid sequence at least 95% identical to the amino acid sequence set forth in SEQ ID NO: 2.


SEQUENCE LISTING (32 sequences)

SEQ ID NO: 1 - DNA, 1549 bp, Homo sapiens
  CDS (68)..(1105)
ggggtggaggagaagtggagtttggaagttcaggggcacaggggcacaggcccacgactgcagcgggatggaccagtactgcatcctgggccgcatcggggagggcgcccacggcatcgtcttcaaggccaagcacgtggagactggcgagatagttgccctcaagaaggtggccctaaggcggttggaagacggcttccctaaccaggccctgcgggagattaaggctctgcaggagatggaggacaatcagtatgtggtacaactgaaggctgtgttcccacacggtggaggctttgtgctggcctttgagttcatgctgtcggatctggccgaggtggtgcgccatgcccagaggccactagcccaggcacaggtcaagagctacctgcagatgctgctcaagggtgtcgccttctgccatgccaacaacattgtacatcgggacctgaaacctgccaacctgctcatcagcgcctcaggccagctcaagatagcggactttggcctggctcgagtcttttccccagacggcagccgcctctacacacaccaggtggccaccaggtggtaccgagcccccgagctcctgtatggtgcccgccagtatgaccagggcgtcgatctgtggtctgtgggctgcatcatgggggagctgttgaatgggtccccccttttcccgggcaagaacgatattgaacagctttgctatgtgcttcgcatcttgggcaccccaaaccctcaagtctggccggagctcactgagctgccggactacaacaagatctcctttaaggagcaggtgcccatgcccctggaggaggtgctgcctgacgtctctccccaggcattggatctgctgggtcaattccttctctaccctcctcaccagcgcatcgcagcttccaaggctctcctccatcagtacttcttcacagctcccctgcctgcccatccatctgagctgccgattcctcagcgtctagggggacctgcccccaaggcccatccagggcccccccacatccatgacttccacgtggaccggcctcttgaggagtcgctgttgaacccagagctgattcggcccttcatcctggaggggtgagaagttggccctggtcccgtctgcctgctcctcaggaccactcagtccacctgttcctctgccacctgcctggcttcaccctccaaggcctccccatggccacagtgggcccacaccacaccctgccccttagcccttgcgagggttggtctcgaggcagaggtcatgttcccagccaagagtatgagaacatccagtcgagcagaggagattcatggcctgtgctcggtgagccttaccttctgtgtgctactgacgtacccatcaggacagtgagctctgctgccagtcaaggcctgcatatgcagaatgacgatgcctgccttggtgctgcttccccgagtgctgcctcctggtcaaggagaagtgcagagagtaaggtgtccttatgttggaaactcaagtggaaggaagatttggtttggttttattctcagagcc

SEQ ID NO: 2 - protein, 346 aa, Homo sapiens
MDQYCILGRIGEGAHGIVFKAKHVETGEIVALKKVALRRLEDGFPNQALREIKALQEMEDNQYVVQLKAVFPHGGGFVLAFEFMLSDLAEVVRHAQRPLAQAQVKSYLQMLLKGVAFCHANNIVHRDLKPANLLISASGQLKIADFGLARVFSPDGSRLYTHQVATRWYRAPELLYGARQYDQGVDLWSVGCIMGELLNGSPLFPGKNDIEQLCYVLRILGTPNPQVWPELTELPDYNKISFKEQVPMPLEEVLPDVSPQALDLLGQFLLYPPHQRIAASKALLHQYFFTAPLPAHPSELPIPQRLGGPAPKAHPGPPHIHDFHVDRPLEESLLNPELIRPFILEG

SEQ ID NO: 3 - DNA, 21 bp, Artificial Sequence
  Description of Artificial Sequence Primer
ggggtggaggagaagtggagt

SEQ ID NO: 4 - DNA, 25 bp, Artificial Sequence
  Description of Artificial Sequence Primer
ggctctgagaataaaaccaaaccaa

SEQ ID NO: 5 - DNA, 2113 bp, Rattus rattus
tttttaagcatgacaaaaataattttattgttggggggagaactgtattacggggttgcagcactgggaagtagagaaccgccttttagactcatcttaatatattcctatgaaataaaaatcaagctggctgagtcacactggaatctacttaccgagtgaattccaggaaagcctgcttcgaatctgcacagcaggctctggctcactgactaacacccaacatgtggaaagtgtgctttgggaactgcactgcccattgctgggcctcaacttctggacctcactcccatgtgagctctcaagtcccatgaagagcacaggaagtctcagaacccaaatggcccttactagaggcaccatctgggtacctgacccctacgaattaaggccctttccctaacaacaacagacaaggtgcaaatacaggcagaaacacccagggcttttatatgaatctcaaagtctgacccagtgaagccaaagccaaagcatgtctgacttcccagctgtgtccccatatgaagacagcttattctctgtgcttccccgaggtggtgctgtagtatcaatgtccttgaagaggagacagaccctgagaatctctctaaggccaggtagcaccactgagagatggccccagagaagaacacagatggcagtttctgggggctgtgagtctctttctctgctgcggtgaacatcttcatggttctgcctgtcagagcccagccctcaagcacgctaagaagcagaaccgggtgtggtacatatagccatggggaggccatgggggccaatcaggatgaggcagaagagtagctgtctcggtgcacctgggttgagcaggaaggactggaccagcatctcagccctctgggatgaagggccggatcagttctggattcaacagtgactcctcaagaggacgatccacatggaagtcatggacatgggggggccctggatgagccttgggtgtgggtccccctgggcgctgaggaattggcagctcagacggatgggcaggcagaggagctgtgaagaaatactgatgcagaagggcctgggaggctgcaatacgctgtcgtggagggtaaaggaggaactgacccagcaggtccagggcctggtgagaggcatcaggcagcacctcctccaggggcactggtgcctgctccttgaaggagatcttgttgtagtcaggcagctctgtgatctccggccagactcgaggactcggggtacccaggattcgaagcacacagcacagttgttcaatgtcgttttctcccgggaataggggggatccattcaacagctctcccataatacagcctacagcccataggtcaacaccctggtcatactgccgagcaccgtacaggagctcaggagctcggtaccacctggtggccacctgatgtgtgtagaggcgaccaccatctggagagaagacccgggccagtccaaagtcagctatcttgagctggcctgaggcactgatgagcaggttggcaggcttcaggtcccgatgcacgatgttgttggcatggcaaaatgcaacacctttgagcagcatctgcaggtagctcttaacctgtgctggggctagtggcctctgggcatggcgcaccacctccgccaggtccgacagcataaactcgaaagccagcacaaagcctgcaccatgtgggaacacagccttcagctgtaccacatattgactgtcctcgatctcctgcagagccttgatttccctgagggcctggttaggaataccatcctccagccgccgcagggccaccttcttgagggcaacgatctcgccagtctctacgtgcttggctttaaagacgatgccgtgggcgccctccccgatgcgaccgaggatgcaatactgatccatgtcggcccgcggcgcccagatcccacactgcggggatctaacctagcaaggagcctgcaagcgttgcctagcaataacgcgtctagcagctgctccataaagcacgtgaccggaaagaccggtgctgagctcagtcaggaatctgcatgccctctaggcagcacctttccgctgccttcagcagtcactccatcttgaactaac

SEQ ID NO: 6 - protein, 346 aa, Rattus rattus
MDQYCILGRIGEGAHGIVFKAKHVETGEIVALKKVALRRLEDGIPNQALREIKALQEIEDSQYVVQLKAVFPHGAGFVLAFEFMLSDLAEVVRHAQRPLAPAQVKSYLQMLLKGVAFCHANNIVHRDLKPANLLISASGQLKIADFGLARVFSPDGGRLYTHQVATRWYRAPELLYGARQYDQGVDLWAVGCIMGELLNGSPLFPGENDIEQLCCVLRILGTPSPRVWPEITELPDYNKISFKEQAPVPLEEVLPDASHQALDLLGQFLLYPPRQRIAASQALLHQYFFTAPLPAHPSELPIPQRPGGPTPKAHPGPPHVHDFHVDRPLEESLLNPELIRPFIPEG

SEQ ID NO: 7 - DNA, 1488 bp, Homo sapiens
  CDS (68)..(1426)
ggggtggaggagaagtggagtttggaagttcaggggcacaggggcacaggcccacgactgcagcgggatggaccagtactgcatcctgggccgcatcggggagggcgcccacggcatcgtcttcaaggccaagcacgtggagactggcgagataattgccctcaagaaggtggccctaaggcggttggaagacggcttccctaaccaggccctgcgggagattaaggctctgcaggagatggaggacaatcagtatgtggtacaactgaaggctgtgttcccacacggtggaggctttgtgctggcctttgagttcatgctgtcggatctggccgaggtggtgcgccatgcccagaggccactagcccaggcacaggtcaagagctacctgcagatgctgctcaagggtgtcgccttctgccatgccaacaacattgtacatcgggacctgaaacctgccaacctgctcatcagcgcctcaggccagctcaagatagcggactttggcctggctcgagtcttttccccagacggcagccgcctctacacacaccaggtggccaccaggtctgtgggctgcatcatgggggagctgttgaatgggtccccccttttcccgggcaagaacgatattgaacagctttgctatgtgcttcgcatcttgggcaccccaaaccctcaagtctggccggagctcactgagctgccggactacaacaagatctcctttaaggagcaggtgcccatgcccctggaggaggtgctgcctgacgtctctccccaggcattggatctgctgggtcaattccttctctaccctcctcaccagcgcatcgcagcttccaaggctctcctccatcagtacttcttcacagctcccctgcctgcccatccatctgagctgccgattcctcagcgtctagggggacctgcccccaaggcccatccagggcccccccacatccatgacttccacgtggaccggcctcttgagggagtcgctgttgaacccagagctgattcggcccttcatcctggaggggtgagaagttggccctggtcccgtctgcctgctcctcaggaccactcagtccacctgttcctctgccacctgcctggcttcaccctccaaggcctccccatggccacagtgggcccacaccacaccttgccccttagcccttgcgagggttggtctcgaggcagaggtcatgttcccagccaagagtatgagaacatccagtcgagcagaggagattcatggcctgtgctcggtgagccttaccttctgtgtgctactgacgtacccatcaggacagtgagctctgctgccagtcaaggcctgcatatgcagaatgacgatgcctgccttggtgctgcttcccccgagtgctgcctcctggtcaaggagaagtgcagagagtaaggtgtccttatgttggaaactcaagtggaaggaagatttggtttggttttattctcagagcc

SEQ ID NO: 8 - protein, 452 aa, Homo sapiens [sequence omitted]

SEQ ID NO: 9 - DNA, 30 bp, Artificial Sequence
  Description of Artificial Sequence Primer
atggaccagtactgcatcctgggccgcatc

SEQ ID NO: 10 - DNA, 27 bp, Primula cuneifolia
accttactctctgcacttctccttgac

SEQ ID NO: 11 - DNA, 19 bp, Artificial Sequence
  Description of Artificial Sequence Primer
tgccaacctgctgatcagc

SEQ ID NO: 12 - DNA, 19 bp, Artificial Sequence
  Description of Artificial Sequence Primer
agactcgagccaggccaaa

SEQ ID NO: 13 - DNA, 25 bp, Artificial Sequence
  Description of Artificial Sequence Probe
cctcaggccagctcaagatagcgga

SEQ ID NO: 14 - protein, 17 aa, Artificial Sequence
  Description of Artificial Sequence Peptide
PKAHPGPPHIHDFHVDR

SEQ ID NO: 15 - protein, 11 aa, Artificial Sequence
  Description of Artificial Sequence Peptide
YGRKKRRQRRR

SEQ ID NO: 16 - protein, 346 aa, Homo sapiens
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGARMYGVGVDMWAVGCILAELLLRVPFLPGDSDLDQLTRIFETLGTPTEEQWPDMCSLPDYVTFKSFPGIPLHHIFSAAGDDLLDLIQGLFLFNPCARITATQALKMKYFSNRPGPTPGCQLPRPNCPVETLKEQSNPALAIKRKRTEALEQGGLPKKLIF

SEQ ID NO: 17 - protein, 305 aa, Homo sapiens
MDMFQKVEKIGEGTYGVVYKAKNRETGQLVALKKIRLDLEMEGVPSTAIREISLLKELKHPNIVRLLDVVHNERKLYLVFEFLSQDLKKYMDSTPGSELPLHLIKSYLFQLLQGVSFCHSHRVIHRDLKPQNLLINELGAIKLADFGLARAFGVPLRTYTHEVVTLWYRAPEILLGSKFYTTAVDIWSIGCIFAEMVTRKALFPGDSEIDQLFRIFRMLGTPSEDTWPGVTQLPDYKGSFPKWTRKGLEEIVPNLEPEGRDLLMQLLQYDPSQRITAKTALAHPYFSSPEPSPAARQYVLQRFRH

SEQ ID NO: 18 - protein, 298 aa, Homo sapiens
  SITE (37)..(37): Xaa = any amino acid
MENFQKVEKIGEGTYGVVYKARNKLTGEVVALKKIRXDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 19 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 20 - DNA, 19 bp, Artificial Sequence
  Description of Artificial Sequence Primer
ggtggtgtggccaggagtt

SEQ ID NO: 21 - DNA, 16 bp, Artificial Sequence
  Description of Artificial Sequence Primer
tcttgccgggcccact

SEQ ID NO: 22 - DNA, 31 bp, Artificial Sequence
  Description of Artificial Sequence Probe
ttctatgcctgattacaagccaagtttcccc

SEQ ID NO: 23 - DNA, 19 bp, Artificial Sequence
  Description of Artificial Sequence primer
aacgtgcaagcctcggatt

SEQ ID NO: 24 - DNA, 19 bp, Artificial Sequence
  Description of Artificial Sequence Primer
gcccagctcagtacaggca

SEQ ID NO: 25 - DNA, 22 bp, Artificial Sequence
  Description of Artificial Sequence Probe
ttgcaccatccagaggctcccc

SEQ ID NO: 26 - DNA, 30 bp, Artificial Sequence
  Description of Artificial Sequence Primer
cattagagttccagtacagaatactgactg

SEQ ID NO: 27 - DNA, 24 bp, Artificial Sequence
  Description of Artificial Sequence Primer
  Description of Artificial Sequence Primer
acctgaggctttcttaaccacttc

SEQ ID NO: 28 - DNA, 27 bp, Artificial Sequence
  Description of Artificial Sequence Probe
tgctgccttgtgccattttacctccat

SEQ ID NO: 29 - DNA, 57 bp, Artificial Sequence
  Description of Artificial Sequence Primer
tgcggccgcatggactacaaagacgatgacgacaagatggaccagtactgcatcctg

SEQ ID NO: 30 - DNA, 27 bp, Artificial Sequence
  Description of Artificial Sequence Primer
gaattctcacccctccaggatgaaggg

SEQ ID NO: 31 - DNA, 29 bp, Artificial Sequence
  Description of Artificial Sequence Primer
tgcgccgcatggaccagtactgcatcctg

SEQ ID NO: 32 - DNA, 51 bp, Artificial Sequence
  Description of Artificial Sequence Primer
gaattctcacttgtcgtcatcgtctttgtagtccccctccaggatgaaggg